(19) United States
(12) Patent Application Publication
(10) Pub. No.: US 2014/0335153 A1
(43) Pub. Date: Nov. 13, 2014

(54) THIN FILM WITH HIGH LOAD OF ACTIVE INGREDIENT

(71) Applicant: CURE Pharmaceutical Corporation, Oxnard, CA (US)

(72) Inventors: Eric Allen, Camarillo, CA (US); Robert Steven Davidson, Camarillo, CA (US); Jose Bernardo, Oxnard, CA (US)

(73) Assignee: CURE Pharmaceutical Corporation, Oxnard, CA (US)

(21) Appl. No.: 14/069,239

(22) Filed: Oct. 31, 2013

Related U.S. Application Data
(63) Continuation-in-part of application No. 13/890,875, filed on May 9, 2013.

(51) Int. Cl. A61K 9/70; A61K 47/44

(52) U.S. Cl. CPC A61K 9/7015; A61K 47/44
USPC 424/450; 514/262.1; 514/250; 514/167; 514/474; 514/178; 514/329; 514/570; 514/165; 514/471; 514/327; 514/629; 514/253.04; 514/370; 514/282; 424/728; 514/397; 514/263.34; 514/53; 514/563; 514/415

(57) ABSTRACT

A method of preparing a thin film, the method comprising: (a) mixing a lipid, emulsifier, and solvent to provide a uniform first mixture; (b) contacting an active ingredient with the uniform first mixture to provide a thickened second mixture; (c) contacting a binder with the thickened second mixture to provide a slurry; and (d) hot extruding, casting, and condensing the slurry to provide the thin film; or cooling, shearing, mixing, casting, and condensing the slurry to provide the thin film.

### RELATED APPLICATION

This application is a continuation-in-part (CIP) and claims the priority of U.S. application Ser. No. 13/890,875, filed May 9, 2013, the contents of which are incorporated herein in its entirety.

### SUMMARY

In various embodiments, the thin film described herein includes the potential to improve the onset of action, lower the dosing, and enhance the efficacy and safety profile of the active ingredient. All tablet dosage forms, softgels, and liquid formulations primarily enter the blood stream via the gastrointestinal tract, which subjects the drug to degradation from stomach acid, bile, digestive enzymes, and other effects including first-pass liver metabolism. As a result, such formulations often require higher doses and generally have a delayed onset of action. Conversely, the thin film described herein can avoid these issues and yield quicker onsets of action at lower doses.

In various embodiments, the thin film described herein provides for a stable, durable and quick dissolving dosage form.

In various embodiments, the thin film described herein enables suitable dosing accuracy, since every strip is manufactured to contain a relatively precise amount of the active ingredient.

In various embodiments, the thin film described herein can be used for the buccal, sublingual, ocular and/or intravaginal administration of active ingredient. The absorption (e.g., systemic absorption) of active ingredient via this route may be considerably higher, compared to the oral route (e.g., solid oral dosage or liquid form). This may lead to an increased therapeutic index. This may also allow for a lower dosing of active ingredient, which may lead to a lower incidence of adverse side effects.

In various embodiments, the thin film described herein can be used for the buccal, sublingual, ocular and/or intravaginal administration of active ingredient. Various active ingredients may possess a higher stability when administered via this route, compared to an oral administration. For example, various active ingredients (e.g., peptides) degrade in the gastrointestinal (GI) tract, which subjects the active ingredient to degradation from stomach acid, bile, digestive enzymes, and other first-pass effects. These active ingredients may not degrade as much (or as readily) when the administration is buccal, sublingual, ocular and/or intravaginal.

In various embodiments, the thin film described herein not only ensures more accurate administration of active ingredients but also can improve compliance due to the intuitive nature of the dosage form and its inherent ease of administration. Specifically, the thin films described herein can be administered to a mucosal surface without the use of fluid. As such, when a fluid is not readily available, when the patient is not conscious or awake, when the patient is suffering from nausea (e.g., those patients receiving chemotherapy), and/or when the subject experiences dysphagia, the medication can still be administered. These properties are especially beneficial for pediatric, geriatric, psychiatric, and patients suffering from a neurodegenerative disease where proper and complete dosing can be difficult. This can also lead to an increase in patient compliance.

In various embodiments, the thin film described herein has the potential to allow the development of sensitive active ingredient targets that may otherwise not be possible in tablet or liquid formulations.

In various embodiments, the thin film described herein has the potential to employ active ingredients that otherwise have stability or incompatibility limitations (e.g., by themselves or with each other).

The thin films described herein can deliver a convenient, quick-dissolving therapeutic dose contained within an abuse-deterrent film matrix that cannot readily be crushed or injected by patients, and ensures compliance as it rapidly absorbs under the tongue, in the buccal mucosa, and/or is swallowed as it disintegrates in the mouth.

In various embodiments, the thin film described herein can be manufactured to include a relatively high load of active ingredient. For example, the active ingredient can be present in about 25-40 wt. %. Such a relatively high load of active ingredient can be advantageous when the delivery of a high load of active ingredient is needed, especially when conventional thin films cannot be readily manufactured to include such a high load of the active ingredient.

The present invention provides a thin film (e.g., an oral thin film) that includes: (a) solvent, (b) binder, (c) lipid, (d) emulsifier, and (e) active ingredient. The thin film can optionally further include at least one of a flavoring agent, a sweetener, a dye or pigment, a preservative, a powder coating, a bitter blocker, and an absorption enhancer. Additionally, the active ingredient can optionally be at least partially encapsulated by the lipid.

The present invention also provides a thin film (e.g., an oral thin film) that includes: (a) about 2-24 wt. % solvent, (b) about 4-50 wt. % binder, (c) about 0-10 wt. % flavoring agent, (d) about 1-40 wt. % sweetener, (e) about 4-22 wt. % lipid, (f) about 3-22 wt. % emulsifier, (g) about 0-1.0 wt. % dye or pigment, (h) about 0-0.1 wt. % preservative, (i) up to about 65 wt. % active ingredient, and (j) about 0-20 wt. % powder coating.

The present invention also provides a thin film (e.g., an oral thin film) that includes: (a) about 4-12 wt. % solvent, (b) about 10-36 wt. % binder, (c) about 2-4 wt. % flavoring agent, (d) about 6-14 wt. % sweetener, (e) about 10-18 wt. % lipid, (f) about 8-18 wt. % emulsifier, (g) about 0.01-0.04 wt. % dye or pigment, (h) about 0-0.02 wt. % preservative, (i) up to about 40 wt. % active ingredient, and (j) about 5-15 wt. % powder coating. The active ingredient can optionally be at least partially encapsulated by the lipid.

The present invention also provides a thin film (e.g., an oral thin film) that includes: (a) about 7.4 wt. % solvent, (b) about 23 wt. % binder, (c) about 3.7 wt. % flavoring agent, (d) about 8.8 wt. % sweetener, (e) about 13.7 wt. % lipid, (f) about 12.9 wt. % emulsifier, (g) about 0.02 wt. % dye or pigment, (h) about 30.5 wt. % active ingredient, and (i) about 9.2 wt. % powder coating. The active ingredient can optionally be at least partially encapsulated by the lipid.

The present invention also provides a method of administering a thin film described herein to a mucosal surface or wound of an animal (e.g., human). The method includes contacting the mucosal surface or wound of a subject with the thin film.

The present invention also provides a method of administering an active ingredient to a mucosal surface or wound of an animal (e.g., human). The method includes contacting the mucosal surface or wound of a subject with a thin film described herein. For example, the administration can be oral (e.g., buccal or sublingual), and the active ingredient can be locally delivered or systemically delivered.

The present invention also provides a method of treating a disease or disorder in an animal (e.g., human). The method includes contacting the mucosal surface or wound of a subject in need of such treatment with a thin film described herein. For example, the administration can be oral (e.g., buccal or sublingual), and the active ingredient can be locally delivered or systemically delivered.

The present invention also provides a method of introducing to a liquid (or liquid containing substance) a thin film described herein. The method includes contacting the liquid (or liquid containing substance) with the thin film, and allowing it to dissolve.

The present invention also provides a method of preparing a liquid dosage form of an active. The method includes contacting a liquid (or liquid containing substance) with a thin film described herein, and allowing it to dissolve.

The present invention also provides a method of flavoring a beverage or food product. The method includes contacting the beverage or food product with a thin film described herein, and allowing it to dissolve.

The present invention also provides a method of adding to a beverage or food product at least one of a sweetener, electrolyte, nutrient, nutraceutical, active ingredient, vitamin, and protein. The method includes contacting the beverage or food product with a thin film described herein, and allowing it to dissolve.

The present invention also provides a method of preparing a thin film (e.g., an oral thin film). The method includes: (a) mixing a lipid, emulsifier, and solvent to provide a uniform first mixture; (b) contacting an active ingredient with the uniform first mixture to provide a thickened second mixture; (c) contacting a binder with the thickened second mixture to provide a slurry; and (d) hot extruding, casting, and condensing the slurry to provide the thin film; or cooling, shearing, mixing, casting, and condensing the slurry to provide the thin film.

The present invention also provides a system that includes: (a) multiple thin films (e.g., multiple oral thin films), each in direct contact with at least one other thin film, and each independently described herein; (b) packaging material enclosing the multiple thin films; and (c) printed indicia located on the packaging material; wherein the multiple thin films do not readily stick to another.

### BRIEF DESCRIPTION OF THE FIGURES

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates a method of preparing an oral thin film, in various embodiments.

FIG. 2 illustrates a SEM micrograph of a multilamellar liposome structure made up of a set of concentric liposomal spheres, in various embodiments.

FIG. 3 illustrates a variety of unilamellar and multilamellar liposomal structures, in various embodiments.

FIG. 4 illustrates a diagrammatic representation of a unilamellar liposome, in various embodiments.

### DETAILED DESCRIPTION

Reference will now be made in detail to certain claims of the invention, examples of which are illustrated in the accompanying drawings. While the invention will be described in conjunction with the enumerated claims, it will be understood that they are not intended to limit those claims. On the contrary, the invention is intended to cover all alternatives, modifications, and equivalents, which can be included within the scope of the invention as defined by the claims.

References in the specification to “one embodiment,” “an embodiment,” “an example embodiment,” and the like, indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one of ordinary skill in the art to affect such feature, structure, or characteristic in connection with other embodiments whether or not explicitly described.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods of manufacturing described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

“Oral thin film,” “OTF,” “oral dissolving film,” “oral drug strip,” “oral thin film,” “thin film,” “orally dissolvable film strip,” or “oral strip” refers to a product used to administer active ingredients via absorption in the mouth (buccally or sublingually), the stomach (gastrically), and/or via the small intestines (enterically). The OTF is edible and pharmaceutically acceptable. A film is prepared typically using hydrophilic polymers that rapidly dissolves on the tongue, palatine tissue, or buccal cavity, delivering the active ingredient to the systemic circulation via dissolution when contact with liquid is made. The OTF (or more appropriately “thin film” or “TF”) can also be used to adhere to mucosal tissue (e.g., at least one of mouth, nose, eye, vagina, and rectum), thereby locally delivering the active ingredient(s). As such, it is appreciated that those of skill in the art understand that reference to a thin film for use with mucosal tissue, such as nose, eye, vagina, and rectum, as an “oral thin film” or OTF is appropriate and acceptable.

The term “film” includes thin films and sheets, in any shape, including rectangular, square, or other desired shape. The films described herein may be any desired thickness and size such that it may be placed into the oral cavity of the user. For example, the films may have a relatively thin thickness of from about 0.1 to about 10 mils, or they may have a somewhat thicker thickness of from about 10 to about 30 mils. For some films, the thickness may be even larger, i.e., greater than about 30 mils. In addition, the term “film” includes single-layer compositions as well as multi-layer compositions, such as laminated films. The composition in its dried film form can effectively maintain a relatively uniform distribution of components through the application of controlled drying of the film. For example, the film can have no more than a 20%, 10%, 5%, or 1% variance of the active ingredient, per unit area of the film.

The substances can be selected in an amount such that a desired dissolution rate can be targeted. Upon contact with mucosal tissue (including, e.g., oral mucosa) the TF will completely dissolve within the desired period of time. The period of time will vary but in reference to the oral cavity, the period of time will typically be within about 30-300 seconds.

Dissolving films generally fall into three main classes: fast dissolving, moderate dissolving and slow dissolving. Fast dissolving films generally dissolve in about 1 second to about 30 seconds. Moderate dissolving films generally dissolve in about 1 to about 30 minutes, and slow dissolving films generally dissolve in more than 30 minutes.

The thin film can be manufactured in a manner, employing the ingredients described herein, such that any one or more of the desired pharmacokinetic metrics (e.g., dose, area under the curve, peak plasma concentration, dosing intervals, time to reach peak plasma concentration, clearance, bioavailability, etc.) are achieved. For example, the thin film can be manufactured such that the thin film provides for an immediate release (IR), controlled release (CR), modified release (MR), extended release (ER), or combination thereof, of active ingredient. This can be advantageous in those embodiments wherein multiple active ingredients are employed, each having different chemical and/or physical properties (e.g., pharmacokinetics, absorption kinetics, stability, solubility, bioavailability, etc.). The thin films described herein therefore possess the potential to allow the development of sensitive drug targets that may otherwise not be feasible in tablet or liquid formulations.

“Multiple” refers to two or more (e.g., 2, 3, 4, 5, 6, etc.).

“Solvent” refers to a substance capable of dissolving another substance (a solute), resulting in a solution. When one substance is dissolved into another, a solution is formed. This is opposed to the situation when the compounds are insoluble like sand in water. In solution, all of the ingredients are uniformly distributed at a molecular level and no residue remains. The mixing is referred to as miscibility, whereas the ability to dissolve one compound into another is known as solubility. However, in addition to mixing, both substances in the solution interact with each other. When something is dissolved, molecules of the solvent arrange themselves around molecules of the solute. Heat is involved and entropy is increased making the solution more thermodynamically stable than the solute alone. This arrangement is mediated by the respective chemical properties of the solvent and solute, such as hydrogen bonding, dipole moment and polarizability.

In particular reference to the thin films described herein, the solvent will typically dissolve, but may also suspend, the active ingredient and other substances present in the OTF. During the condensing step, much (if not all) of the solvent can be removed. However, any solvent remaining will become an integral part of the OTF.

“Binder” refers to any material or substance that holds or draws other materials together to form a cohesive whole. Liquid binders are added to a dry substance in order to draw it together in such a way that it maintains a uniform consistency. The binder can also add mucoadhesion to the OTF.

The thin film described herein can optionally further include a mucoadhesive agent. The mucoadhesive agent, when placed in the oral cavity in contact with the mucosa therein, adheres to the mucosa. The mucoadhesive agent is especially effective in transmucosal delivery of the active ingredient, as the mucoadhesive agent permits a close and extended contact of the composition with the mucosal surface by promoting adherence of the composition or drug to the mucosa, and facilitates the release of the active ingredient from the composition. The mucoadhesive agent can be a polymeric compound, such as a cellulose derivative but it may be also a natural gum, alginate, pectin, or such similar polymer. The concentration of the mucoadhesive agent in the coating, such as a powder matrix coating, may be adjusted to vary the length of time that the film adheres to the mucosa or to vary the adhesive forces generated between the film and mucosa. The mucoadhesive agent may adhere to oral mucosa or to mucosa or tissue in other parts of the body, including the mouth, nose, eyes, vagina, and rectum.

Mucoadhesive agents include, e.g., carboxymethyl cellulose, polyvinyl alcohol, polyvinyl pyrrolidone (povidone), sodium alginate, methyl cellulose, hydroxypropyl cellulose, hydroxypropyl methyl cellulose, polyethylene glycols, carbopols, polycarbophils, carboxyvinyl copolymers, propylene glycol alginate, alginic acid, methyl methacrylate copolymers, tragacanth gum, guar gum, karaya gum, ethylene vinyl acetate, dimenthylpolysiloxanes, polyoxyalkylene block copolymers, pectin, chitosan, carrageenan, xanthan gum, gellan gum, locust bean gum, and hydroxyethylmethacrylate copolymers.

“Lipid” refers to a group of naturally occurring molecules that include fats, waxes, sterols, fat-soluble vitamins (such as vitamins A, D, E, and K), monoglycerides, diglycerides, triglycerides, phospholipids, and others. “Lipid” may also refer to ethoxylated fatty alcohols such as oleth-10 and laureth-10 and mixtures of ethoxylated mono and diglycerides such as PEG-16 macadamia glycerides and PEG-10 sunflower glycerides. The compounds are hydrophobic or amphiphilic small molecules. The amphiphilic nature of some lipids allows them to form structures such as vesicles, liposomes, or membranes in an aqueous environment. Biological lipids originate entirely or in part from two distinct types of biochemical subunits or “building-blocks”: ketoacyl and isoprene groups. Using this approach, lipids may be divided into eight categories: fatty acids, glycerolipids, glycerophospholipids, sphingolipids, saccharolipids, and polyketides (derived from condensation of ketoacyl subunits); and sterol lipids and prenol lipids (derived from condensation of isoprene subunits).

Although the term lipid is sometimes used as a synonym for fats, fats are a subgroup of lipids called triglycerides. Lipids also encompass molecules such as fatty acids and their derivatives (including tri-, di-, monoglycerides, and phospholipids), as well as other sterol-containing metabolites such as cholesterol.

In particular reference to the thin films described herein, the lipids facilitate for the formation of liposomes and micelles.

Liposomes are spherical, lipid bilayer vesicles made from amphiphilic lipids. Typically these are phospholipids that are comprised of a glycerin core with two fatty acid esters, and a phosphate group bound to an additional polar group. These phospholipids thus have two lipophilic tails and a single hydrophilic head. Single fatty acid chains with a single hydrophilic head, like typical soap and detergent molecules, will preferentially form unilayer micelles and not bilayer liposomal structures.

Liposomes can be unilamellar, with a single lipid bilayer making a single sphere. They can also form various multilamellar structures. Multilamellar structures can be concentric spheres, or a sphere containing multiple unilamellar spheres within. All combinations of the above have been documented.

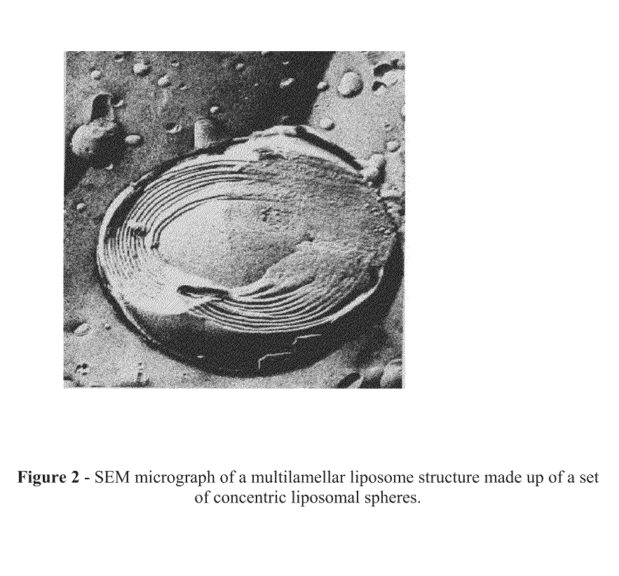

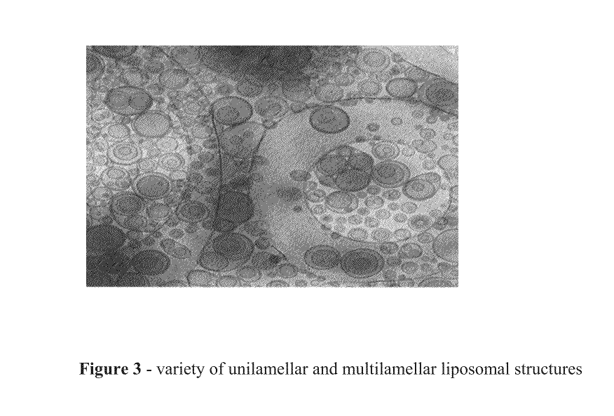

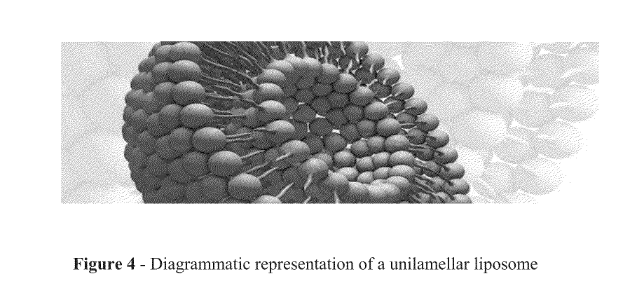

FIG. 2 is a SEM micrograph of a multilamellar liposome structure made up of a set of concentric liposomal spheres. FIG. 3 illustrates a variety of unilamellar and multilamellar liposomal structures. FIG. 4 is a diagrammatic representation of a unilamellar liposome. Note that as the distance from the center of the liposome increases, the density of the atoms decreases. This leads to tight grouping of the polar heads on the interior surface of the lipid bilayer and a looser grouping of polar heads and tails on the outer surface. The addition of non-polar lipids, like triacyl glycerides (standard fats), cholesterol, or tocopherol, can stabilize the bilayer structure by filling in gaps that the amphiphilic lipids by themselves cannot easily fill.

The interior aqueous capsule in the center of a unilamellar liposome can contain water—as well as water soluble atoms or molecules. Likewise, the aqueous layers between the lipid bilayers of a multilamellar liposome structure can contain the same water soluble atoms or molecules as the center capsule. The interstitial aqueous environment exterior to the liposomes can contain the same or different aqueous solutions.

The interior of the lipid bilayer is an environment that acts as an organic solvent, and it will hold lipophilic molecules in solution within the layer. Thus, liposomes contain both lipophilic and hydrophilic solvation environments and can act as carriers of various aqueous and organic molecules.

Liposomes in close proximity to one another can merge to form larger liposome structures. When the interstitial aqueous phase is partially or completely evaporated, liposomes will necessarily come into very close contact with each other. Adding heat can make a liposome's membrane less rigid and more apt to merge with neighboring liposomes.

Liposomes will form when amphiphilic phospholipids are high-shear stirred in an aqueous environment. If fats or other organic materials are included in the process, then they become part of the lipid bilayers that make up the liposome's bilayer membrane. Material dissolved in the aqueous phase will become incorporated into the aqueous phases of the liposomal structures as well. High shear mixing yields predominantly unilamellar liposomes. Low shear yields various combinations of unilamellar and multilamellar liposomal structures.

“Encapsulated” refers to the enclosure of a first compound (e.g., active ingredient) by a second compound (e.g., lipid). As described herein, the second compound (e.g., lipid) can encapsulate the first compound (e.g., active ingredient) by the formation of liposomes and/or micelles. One advantage of the encapsulation of the thin films described herein is the ability to employ bitter substances (e.g., bitter active ingredients), while having the bitter flavor of those substances be at least partially masked.

“Emulsifier” refers to a substance capable of forming or promoting an emulsion. An emulsion is a mixture of two or more liquids that are normally immiscible (nonmixable or unblendable). Emulsions are part of a more general class of two-phase systems of matter called colloids. Although the terms colloid and emulsion are sometimes used interchangeably, emulsion should be used when both the dispersed and the continuous phase are liquids. In an emulsion, one liquid (the dispersed phase) is dispersed in the other (the continuous phase). Examples of emulsions include vinaigrettes, milk, mayonnaise, and some cutting fluids for metal working. The photo-sensitive side of photographic film is an example of a colloid.

In particular reference to the thin films described herein, the emulsifier promotes the separation of phases (e.g., aqueous and lipids), while allowing them to be mixed.

“Flavoring agent” refers to a substance capable of providing a flavor. In addition to providing a palatable and pleasurable factor to the user, the flavoring agent can also mask undesirable flavors present in the OTF. The flavoring agent can include natural flavoring agents (e.g., extracts)

“Flavor extract” refers to a flavoring agent obtained by extracting a part of a raw material, e.g., animal or plant material, often by using a solvent such as ethanol or water. The majority of natural essences are obtained by extracting the essential oil from the blossoms, fruit, roots, etc., or the whole plants, through four techniques: expression (when the oil is very plentiful and easily obtained, as in lemon peel), absorption (generally accomplished by steeping in alcohol, as vanilla beans), maceration (used to create smaller bits of the whole, as in making peppermint extract, etc.), and distillation (used with maceration, but in many cases, it requires expert chemical knowledge and the erection of costly stills).

Flavoring agents can include breath freshening compounds like menthol, spearmint, and cinnamon, coffee beans, other flavors or fragrances such as fruit (e.g., cherry, orange, grape, etc.) flavors, especially those used for oral hygiene, as well as actives used in dental and oral cleansing such as quaternary ammonium bases. The effect of flavors may be enhanced using flavor enhancers like tartaric acid, citric acid, vanillin, or the like.

“Sweetener” refers to a substance capable of providing a palatable and pleasurable factor to the user, and/or capable of masking undesirable flavors present in the OTF. The sweetener can include one or more artificial sweeteners, one or more natural sweeteners, or a combination thereof.

Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides.

Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

“Amino acid” refers to the residues of the natural amino acids (e.g. Ala, Arg, Asn, Asp, Cys, Glu, Gln, Gly, His, Hyl, Hyp, Ile, Leu, Lys, Met, Phe, Pro, Pyl, Sec, Ser, Thr, Trp, Tyr, and Val) in D or L form, as well as unnatural amino acids (e.g. phosphoserine, phosphothreonine, phosphotyrosine, gamma carboxyglutamate; hippuric acid, octahydroindole-2-carboxylic acid, statine, 1,2,3,4,-tetrahydroisoquinoline-3-carboxylic acid, penicillamine, ornithine, citruline, α methyl-alanine, para-benzoylphenylalanine, phenylglycine, propargylglycine, sarcosine, and tert-butylglycine). The term also includes natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Third Edition, Wiley: New York, 1999, and references cited therein).

“Bitter blocker” refers to a substance capable of blocking or diminishing the bitter taste of another substance.

“Palatable” refers to a substance (e.g., oral thin film) being relatively acceptable or agreeable to the palate or taste (e.g., sweet or savory), and in some cases to the olfactory nerves.

“Dye or pigment” or “coloring agent” refers to a substance that imparts coloring and/or aesthetic appearance to the OTF. A dye is a colored substance that has an affinity to the substrate to which it is being applied. The dye is generally applied in an aqueous solution, and requires a mordant to improve the fastness of the dye on the fiber. A pigment is a material that changes the color of reflected or transmitted light as the result of wavelength-selective absorption. This physical process differs from fluorescence, phosphorescence, and other forms of luminescence, in which a material emits light. Both dyes and pigments appear to be colored because they absorb some wavelengths of light more than others. In contrast with a dye, a pigment generally is insoluble, and has no affinity for the substrate. Some dyes can be precipitated with an inert salt to produce a lake pigment, and based on the salt used they could be aluminum lake, calcium lake or barium lake pigments.

One or more dyes, pigments, and coloring agents can be employed in the manufacture of the thin film, such that the thin film has the desired color. Suitable colors include, e.g., white, black, yellow, blue, green, pink, red, orange, violet, indigo, and brown. In specific embodiments, the color of the thin film can indicate the contents (e.g., one or more active ingredients) contained therein. For example, the thin film can include one or more sweeteners as indicated by the color of the thin film. Specifically, the thin film can be blue, as an indication that the active ingredient includes aspartame (marketed as Equal®). Alternatively, the thin film can be blue, as an indication that the active ingredient includes sildenafil citrate (marketed as Viagra®). The thin film can be pink, as an indication that the active ingredient includes saccharine. The thin film can be yellow, as an indication that the active ingredient includes sucralose (marketed as Splenda®). Alternatively, the thin film can be yellow, as an indication that the active ingredient includes saccharine (marketed as Sugar Twin®). The thin film can be green, as an indication that the active ingredient includes stevia. The thin film can be black, as an indication that the active ingredient includes cyclamates. The thin film can be brown, as an indication that the active ingredient includes brown sugar. The thin film can be white, as an indication that the active ingredient includes white sugar.

“Preservative” refers to an agent that extends the storage life of food and non-food products by retarding or preventing deterioration of flavor, odor, color, texture, appearance, nutritive value, or safety. A preservative need not provide a lethal, irreversible action resulting in partial or complete microbial cell destruction or incapacitation. Sterilants, sanitizers, disinfectants, sporicides, viracides and tuberculocidal agents provide such an irreversible mode of action, sometimes referred to as “bactericidal” action. In contrast, a preservative can provide an inhibitory or bacteriostatic action that is reversible, in that the target microbes can resume multiplication if the preservative is removed. The principal differences between a preservative and a sanitizer primarily involve mode of action (a preservative prevents growth rather than killing microorganisms) and exposure time (a preservative has days to months to act whereas a sanitizer has at most a few minutes to act).

“Powder coating” refers to a substance that when used on the external surface of an OTF, prevents, minimizes and/or mitigates the likelihood that the OTF will stick to another adjoining OTF once packaged and/or manufacturing equipment. As such, the powder coating can serve as a processing aid. The powder coating can also provide a vehicle for additional flavoring. The size of the substances present in the powder coating can vary as desired, but will typically be in the range of about 1 μm to about 100 μm. In some embodiments, an active ingredient is located in the powder coating. In further embodiments, the powder coating can augment the dissolution rate of the active ingredient located therein.

“Tensile strength” refers to the maximum stress that a material can withstand while being stretched or pulled before failing or breaking Tensile strength is the opposite of compressive strength and the values can be quite different. Tensile strength is defined as a stress, which is measured as force per unit area. For some non-homogeneous materials (or for assembled components) it can be reported just as a force or as a force per unit width. In the SI system, the unit is the pascal (Pa) (or a multiple thereof, often megapascals (MPa), using the mega-prefix); or, equivalently to pascals, newtons per square meter (N/m²). The customary unit is pounds-force per square inch (lbf/in² or psi), or kilo-pounds per square inch (ksi, or sometimes kpsi), which is equal to 1000 psi; kilo-pounds per square inch are commonly used for convenience when measuring tensile strengths. Typically, the testing involves taking a small sample with a fixed cross-section area, and then pulling it with a controlled, gradually increasing force until the sample changes shape or breaks.

“Pliable” refers to the ability of an article to readily bend, be flexible, or to be supple.

“Non-sticky” refers to an article (e.g., thin film) not having the property of readily adhering or sticking to another surface (e.g., another article, manufacturing equipment, packaging material, the user, etc.).

“Soft” refers to an article being relatively smooth and agreeable to the touch; not rough or coarse. Such an article will be capable of producing agreeable sensations, pleasant or comfortable, upon contact with an animal such as a human.

“Chewable configuration” refers to an article being manufactured in such a manner and with ingredients, that it possesses a configuration capable of being readily chewed by an animal, such as a human.

“Malleable configuration” refers to refers to an article being manufactured in such a manner and with ingredients, that it possesses a configuration capable of being readily shaped or changed in form (e.g., folded, bent, rolled, twisted, flexed, etc.) without breaking.

“Ductile property” refers to the ability of an article (e.g., thin film) being readily shaped or changed in form (e.g., folded, bent, rolled, twisted, flexed, etc.) without breaking.

“Mixing” refers to the act of combining, uniting, and/or joining multiple substances, into one mass, collection, or assemblage (e.g., slurry), generally with a thorough and continuous contacting of the constituents.

“Blending” refers to the act of mixing that employs equipment typically referred to as a blender, or any device capable of blending a mixture. The mixing can provide a relatively smooth mixture, where the constituents are inseparable. When used in the context of “high shear blending”, the blender has sharp edged blades and is used at high speed (1000-10,000 rpm).

“Mixture” refers to the mass, collection, or assemblage (e.g., slurry) obtained from the act of mixing.

“Cooling” refers to the act of removing or transferring a sufficient amount of energy (e.g., thermal energy), within a suitable period of time, such that a decrease in temperature is experienced.

“Shearing” refers to the act of blending using sharp edged blending/mixing blades.

“Slurry” refers to a relatively viscous mixture, but it flows freely. Upon condensing, the slurry will form a film.

As used herein, “contacting” refers to the act of touching, making contact, or of bringing substances into immediate proximity.

“Hot extruding” refers to the act of extruding an article, while the article is currently being heated, or was previously heated, such that the article is at an elevated temperature during the extruding process. The extruding process typically includes forcing the article through a die, thereby obtaining a desired cross-section.

“Casting” or “film casting” refers to the act of removing liquid (e.g., water and/or solvent) from a mixture (e.g., slurry), such that a film is produced.

“Condensing” refers to the act of removing liquid (e.g., water and/or solvent).

“Heating” refers to the act of applying or transferring a sufficient amount of energy (e.g., thermal energy), within a suitable period of time, such that a rise in temperature is experienced.

“Conductive heat transfer” or “conduction” refers to the transfer of heat from one condensed material into another condensed material that does not involve bulk motion within either of the condensed media.

“Radiative heat transfer” or “radiation” refers to the transfer of heat from one article to another by way of electromagnetic means, usually by infrared radiation, but can also be microwave radiation.

“Convective heat transfer” or “convection” refers to the transfer of heat from one article to another, by the movement of fluids. Convection is usually the dominant form of heat transfer in liquids and gases. Although often discussed as a distinct method of heat transfer, convective heat transfer involves the combined processes of conduction (heat diffusion) and advection (heat transfer by bulk fluid flow).

Convection can be “forced” by movement of a fluid by means other than buoyancy forces (for example, a water pump in an automobile engine). In some cases, natural buoyancy forces alone are entirely responsible for fluid motion when the fluid is heated, and this process is called “natural convection.” An example is the draft in a chimney or around any fire. In natural convection, an increase in temperature produces a reduction in density, which causes fluid motion due to pressures and forces when fluids of different densities are affected by gravity (or any g-force). For example, when water is heated on a stove, hot water from the bottom of the pan rises, displacing the colder denser liquid which falls. After heating has stopped, mixing and conduction from this natural convection eventually result in a nearly homogeneous density, and even temperature.

Two types of convective heat transfer can be distinguished: free or natural convection (passive) and forced convection (active). Active convection occurs when a fluid is forced to flow over the surface by an external source such as fans, by stirring, and pumps, creating an artificially induced convection current. Passive convention occurs when fluid motion is caused by buoyancy forces that result from the density variations due to variations of temperature in the fluid. In the absence of an external source, when the fluid is in contact with a hot surface, its molecules separate and scatter, causing the fluid to be less dense. As a consequence, the fluid is displaced while the cooler fluid gets denser and the fluid sinks Thus, the hotter volume transfers heat towards the cooler volume of that fluid. Familiar examples are the upward flow of air due to a fire or hot object and the circulation of water in a pot that is heated from below.

“In vacuo” refers to under vacuum. The vacuum can be a partial vacuum or a complete vacuum. Typically, the vacuum will be a partial vacuum (e.g., a reduced pressure) such as, e.g., a pressure of less than about 30 mm mercury (Hg). Specifically, the reduced pressure can be less than about 29 mm mercury (Hg). More specifically, the reduced pressure can be about 10 to about 29 mm of mercury (Hg).

“Packaging material” refers to those materials and substances employed to package the product (e.g., thin film). Such materials are widely known to those of skill in the art.

“Enclosing” refers to the packaging materials containing or holding the product (e.g., thin film) by surrounding the product with the packaging material. The packaging materials can partially surround the product, or can completely surround the product. Typically, to ensure safety (e.g., no tampering with product) and freshness, the packaging materials will completely surround the product. For example, the packaging materials can form a relatively vapor impermeable enclosure of the product.

“Printed indicia” refers to a marking, image, text, and/or symbol located on the surface of the packaging material. The indicia can be placed on the surface of the packaging material by any suitable means (e.g., ink printing, laser printing, etc.). The indicia can include, e.g., a printed message or instructions, list of ingredients (active and inactive), weight of product, manufacturer name and address, manufacturer trademark, etc. The indicia can be placed on the surfaces of the thin film itself and can include information such as manufacturer trademark, weight of product, weight of active ingredients, manufacturer name, serial numbers, lot numbers, etc.

The thin films described herein can be perforated. “Perforated” refers to the one or more holes, apertures or scores existing along a line to facilitate separation. Perforations on the thin films allow the user to conveniently administer smaller dosages of the active ingredient. This is especially useful, for example, when the patient is a child, who should receive a smaller dosage. Accurate dosing can be metered, e.g., by the weight, size, age, etc. of the patient.

As used herein, “active ingredient” refers to a therapeutic agent and includes any substance, other than food, used in the prevention, diagnosis, alleviation, treatment, or cure of a disease or disorder. Stedman's Medical Dictionary, 25th Edition (1990). The substance can be taken by mouth; injected into a muscle, the skin, a blood vessel, or a cavity of the body; or topically applied. Mosby's Medical, Nursing & Allied Health Dictionary, 5th Edition (1998). The agent can include any substance disclosed in at least one of: The Merck Index, 14th Edition (2006); Pei-Show Juo, Concise Dictionary of Biomedicine and Molecular Biology, (1996); U.S. Pharmacopeia Dictionary, 2000 Edition; Physician's Desk Reference, 2010 Edition; Orange Book: Approved Drug Products with Therapeutic Equivalence Evaluations (April 2013); and Approved Animal & Vetinary Drug Products (Green Book) (January 2013). The term active ingredient includes, e.g., prescription and over the counter active pharmaceutical ingredients (e.g., small molecules, macrocycles, peptides, etc.), vitamins, nutraceuticals, supplements (e.g., dietary, nutritional, and herbal), cosmetics, and biologicals.

As used herein, the term “vitamin” refers to an organic compound required by an organism as a vital nutrient in limited amounts. An organic chemical compound (or related set of compounds) is called a vitamin when it cannot be synthesized in sufficient quantities by an organism, and must be obtained from the diet. Thus, the term is conditional both on the circumstances and on the particular organism. For example, ascorbic acid (Vitamin C) is a vitamin for humans, but not for most other animals, and biotin and vitamin D are required in the human diet only in certain circumstances. Examples of human vitamins include Vitamin A (e.g., retinol, retinal, and four carotenoids including beta carotene), Vitamin B1 (thiamine), Vitamin B2 (riboflavin), Vitamin B3 (e.g., niacin and niacinamide), Vitamin B5 (pantothenic acid), Vitamin B6 (e.g., pyridoxine, pyridoxamine, and pyridoxal), Vitamin B7 (biotin), Vitamin B9 (e.g., folic acid and folinic acid), Vitamin B12 (e.g., cyanocobalamin, hydroxocobalamin, and methylcobalamin), Vitamin C (ascorbic acid), Vitamin D (cholecalciferol), Vitamin E (e.g., tocopherols and tocotrienols), and Vitamin K (e.g., phylloquinone, phytonadione, and menaquinones).

As used herein, the term “nutraceutical” refers to a product isolated or purified from food that is generally sold in medicinal forms not usually associated with food. A nutraceutical is demonstrated to have a physiological benefit or provide protection against chronic disease. Such products may range from isolated nutrients, dietary supplements and specific diets to genetically engineered foods, and herbal products. Examples include antioxidants (e.g., pterostilbene from grapes and blueberries; resveratrol from red grape products; flavonoids inside citrus, tea, wine, and dark chocolate foods; and anthocyanins found in berries), substances believed to reduce hypercholesterolemia (e.g., soluble dietary fiber products, such as psyllium seed husk), substances believed to assist in cancer prevention (e.g., broccoli (sulforaphane) and fiddleheads (Matteuccia struthiopteris)), substances believed to improve arterial health (e.g., soy or clover (isoflavonoids)), substances believed to lower the risk of cardiovascular disease (e.g., alpha-linolenic acid from flax or chia seeds, and omega 3 fatty acids in fish oil). Additional nutraceuticals include, e.g., botanical and herbal extracts such as ginseng, garlic oil, etc.

“Therapeutically effective amount” is intended to include an amount of a compound described herein, or an amount of the combination of compounds described herein, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes: (i) preventing a pathologic condition from occurring (e.g. prophylaxis); (ii) inhibiting the pathologic condition or arresting its development; (iii) relieving the pathologic condition; and/or (iv) diminishing symptoms associated with the pathologic condition.

The thin film can be administered, e.g., to a human patient in need of a treatment of a disease or disorder. Selection of the active ingredient(s) within the thin film described herein will be dependent upon the disease or disorder to be treated. The above-mentioned references (e.g., Physician's Desk Reference, 2010 Edition) provide a description of the diseases or disorders that specific active ingredients have been approved for by the U.S. FDA, in the marketing and sale of the product within the United States. As such, a skilled artisan can look to such references for guidance in the selection of the active ingredient(s) to be present within the thin film, based upon the treatment of the specific disease or disorder of particular interest.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The active ingredient(s) can be present in any suitable and appropriate amount, depending upon the desired dosing. For example, in a 100 mg thin film, the active ingredient(s) can be present in an amount of about 0.01-60 mg, about 0.1-50 mg, or about 0.5-40 mg.

Specific classes of active pharmaceutical ingredients (APIs) include, e.g., ace-inhibitors, anti-Alzheimer's agents, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-emetics, anti-histamines, anti-hypertensive drugs, anti-inflammatory agents, anti-lipid agents, anti-manics, anti-migraines, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, anxiolytics, anti-psychotics, appetite stimulants, biological response modifiers, blood modifiers, bone metabolism regulators, bronchodilators, cardiovascular agents, central nervous system stimulates, cholinesterase inhibitors, contraceptives, decongestants, dietary supplements, dopamine receptor agonists, endometriosis management agents, enzymes, erectile dysfunction agents, fertility agents, gastrointestinal agents, H₂-antagonists, homeopathic remedies, hormones, hypercalcemia and hypocalcemia management agents, immunomodulators, immunosuppressives, migraine preparations, motion sickness treatments, muscle relaxants, non-steroidal anti-inflammatories (NSAID's), obesity management agents, osteoporosis preparations, oxytocics, parasympatholytics, parasympathomimetics, prostaglandins, psychotherapeutic agents, respiratory agents, sedatives, serotonin 5-HT3 receptor antagonists, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, anti-obesity drugs, erythropoietic drugs, anti-asthmatics, cough suppressants, mucolytics, DNA and genetic modifying drugs, and combinations thereof.

Additional specific classes of active pharmaceutical ingredients (APIs), by prescribing category and therapeutic use or mechanism of action, are provided in Table A below.

TABLE A

| | |
|---|---|
| PRESCRIBING | THERAPEUTIC USE OR |
| CATEGORY | MECHANISM OF ACTION |

A

| | |
|---|---|
| ACETYLCHOLINE AGONISTS | |
| ACROMEGALY AGENTS | |
| AIDS/HIV ADJUNCT AGENTS | |
| ALCOHOL ABUSE | ALCOHOL DEPENDENCE |
| PREPARATIONS | ALCOHOL WITHDRAWAL |
| ALZHEIMER'S DISEASE | |
| MANAGEMENT | |
| AMYOTROPHIC LATERAL | |
| SCLEROSIS THERAPEUTIC | |
| AGENTS | |
| ANALGESICS | ACETAMINOPHEN & COMBINATIONS |
| | CENTRALLY ACTING ANALGESIC |
| | MISCELLANEOUS ANALGESIC AGENTS |
| | NARCOTICS |
| | NARCOTIC AGONIST- |
| | ANTAGONIST & |
| | COMBINATIONS |
| | NARCOTICS & |
| | COMBINATIONS |
| | NONSTEROIDAL ANTI-INFLAMMATORY |
| | DRUGS (NSAIDS) & COMBINATIONS |
| | SALICYLATES |
| | ASPIRIN & COMBINATIONS |
| | OTHER SALICYLATES & |
| | COMBINATIONS |
| ANESTHETICS | GENERAL ANESTHETICS |
| | LOCAL ANESTHETICS |
| ANTICONVULSANTS | BARBITURATES |
| | BENZODIAZEPINES |
| | GABA ANALOGUES |
| | HYDANTOINS |
| | MISCELLANEOUS ANTICONVULSANTS |
| | PHENYLTRIAZINES |
| ANTIDIABETIC AGENTS | BIGUANIDES & COMBINATIONS |
| | GLUCOSIDASE INHIBITORS |
| | INSULINS |
| | INTERMEDIATE ACTING |
| | INSULINS |
| | INTERMEDIATE AND RAPID |
| | ACTING INSULIN |
| | COMBINATIONS |
| | LONG ACTING INSULINS |
| | RAPID ACTING INSULINS |
| | MEGLITINIDES |
| | MISCELLANEOUS ANTIDIABETIC AGENTS |
| | SULFONYLUREAS & COMBINATIONS |
| | THIAZOLIDINEDIONES & COMBINATIONS |
| ANTIDOTES | ACETAMINOPHEN ANTAGONISTS |
| | ANTIVENINS |
| | BENZODIAZEPINE ANTAGONISTS |
| | CHELATING AGENTS |
| | CYANIDE |
| | IRON |
| | LEAD |
| | DIGOXIN ANTAGONISTS |
| ANTIFIBROSIS THERAPY, | |
| SYSTEMIC | |
| ANTIHISTAMINES & | |
| COMBINATIONS | |
| ANTI-INFECTIVE AGENTS, | AIDS ADJUNCT ANTI-INFECTIVES |
| SYSTEMIC | AIDS CHEMOTHERAPEUTIC AGENTS |
| | NON-NUCLEOSIDE REVERSE |
| | TRANSCRIPTASE INHIBITORS |
| | & COMBINATIONS |
| | NUCLEOSIDE REVERSE |
| | TRANSCRIPTASE INHIBITORS |
| | & COMBINATIONS |
| | NUCLEOTIDE ANALOGUE |
| | REVERSE TRANSCRIPTASE |
| | INHIBITORS & |
| | COMBINATIONS |
| | PROTEASE INHIBITORS |
| | ANTHELMINTICS |
| | ANTIBIOTICS |
| | AMINOGLYCOSIDES |
| | -LACTAM ANTIOBIOTICS, |
| | MISCELLANEOUS |
| | CEPHALOSPORINS |
| | MACROLIDES & |
| | COMBINATIONS |
| | MISCELLANEOUS |
| | ANTIBIOTICS |
| | PENICILLINS & |
| | COMBINATIONS |
| | QUINOLONES |
| | SULFONAMIDES & |
| | COMBINATIONS |
| | TETRACYCLINES |
| | ANTIFUNGALS |
| | ANTIMALARIAL AGENTS |
| | ANTIPROTOZOAL AGENTS |
| | ANTITUBERCULOSIS AGENTS |
| | ANTIVIRALS |
| | LEPROSTATICS |
| | MISCELLANEOUS ANTI-INFECTIVES |
| | URINARY ANTI-INFECTIVES & |
| | COMBINATIONS |
| ANTI-INFECTIVES, NON- | MISCELLANEOUS, ANTI-INFECTIVES, NON |
| SYSTEMIC | SYSTEMIC |
| ANTINEOPLASTICS | ADJUNCT ANTINEOPLASTIC THERAPY |
| | ALKYLATING AGENTS |
| | MISCELLANEOUS |
| | ALKYLATING AGENTS |
| | NITROGEN MUSTARDS |
| | NITROSOUREAS |
| | ANTIBIOTICS |
| | ANTIMETABOLITES |
| | CYTOTOXIC AGENTS |
| | HORMONAL AGONISTS/ANTAGONISTS |
| | ANTIESTROGENS |
| | ESTROGENS |
| | GONADOTROPIN RELEASING |
| | HORMONE (GNRH) |
| | ANALOGUES |
| | IMMUNOMODULATORS |
| | MISCELLANEOUS |
| | ANTINEOPLASTICS |
| | MULTI-KINASE INHIBITOR |
| | PHOTOSENSITIZING AGENTS |
| | SKIN & MUCOUS MEMBRANE |
| | AGENTS |
| | STEROIDS & COMBINATIONS |
| | TAXOIDS |
| ANTIPARKINSONIAN | CATECHOL-O-METHYLTRANSFERASE |
| AGENTS | INHIBITORS |
| | DOPAMINE AGONISTS |
| | DOPAMINERGIC AGENTS |
| | MONOAMINE OXIDASE INHIBITORS (MAOI) |
| ANTIRHEUMATIC AGENTS | MISCELLANEOUS ANTIRHEUMATIC AGENTS |

B

| | |
|---|---|
| BIOLOGICAL RESPONSE | |
| MODIFIERS | |
| BIOLOGICALS | ALPHA1-PROTEINASE INHIBITOR |
| | ANTITOXINS & ANTIVENINS |
| | IMMUNE SERUMS |
| | MISCELLANEOUS BIOLOGICALS |
| | TOXOIDS |
| | VACCINES |
| BLOOD MODIFIERS | ANTICOAGULANTS |
| | ANTIPLATELET AGENTS |
| | COLONY STIMULATING FACTORS |
| | GRANULOCYTE (G-CSF) |
| | GRANULOCYTE |
| | MACROPHAGE (GM-CSF) |
| | HEMATINICS |
| | ERYTHROPOIESIS |
| | STIMULANTS |
| | FOLIC ACID DERIVATIVES & |
| | COMBINATIONS |
| | IRON & COMBINATIONS |
| | LIVER & COMBINATIONS |
| | MISCELLANEOUS BLOOD |
| | MODIFIERS |
| | HEMOSTATICS |
| | SYSTEMIC HEMOSTATICS |
| | PLASMA FRACTIONS, HUMAN |
| | ALBUMIN |
| | ANTIHEMOPHILIC FACTOR |
| | ANTI-INHIBITOR |
| | COAGULANT COMPLEX |
| | ANTITHROMBIN III |
| | FACTOR IX COMPLEX |
| | IMMUNE GLOBULIN |
| | PLASMA PROTEIN FRACTION |
| | SELECTIVE FACTOR XA |
| | INHIBITOR |
| | THROMBIN INHIBITORS |
| | THROMBOLYTIC AGENTS |
| | VITAMIN K |
| BONE METABOLISM | |
| REGULATORS | |

C

| | |
|---|---|
| CARDIOPROTECTIVE | |
| AGENTS | |
| CARDIOVASCULAR AGENTS | ADRENERGIC BLOCKERS, PERIPHERAL & |
| | COMBINATIONS |
| | ADRENERGIC STIMULANTS, CENTRAL & |
| | COMBINATIONS |
| | ALPHA/BETA ADRENERGIC BLOCKERS |
| | ANGIOTENSIN CONVERTING ENZYME (ACE) |
| | INHIBITORS |
| | ANGIOTENSIN CONVERTING ENZYME (ACE) |
| | INHIBITORS WITH CALCIUM CHANNEL |
| | BLOCKERS |
| | ANGIOTENSIN CONVERTING ENZYME (ACE) |
| | INHIBITORS WITH DIURETICS |
| | ANGIOTENSIN II RECEPTOR ANTAGONISTS |
| | ANGIOTENSIN II RECEPTOR ANTAGONISTS |
| | WITH DIURETICS |
| | ANTIARRHYTHMICS |
| | GROUP I |
| | MISCELLANEOUS |
| | ANTIARRHYTHMICS |
| | ANTIHYPERTENSIVE AGENTS |
| | MISCELLANEOUS |
| | ANTIHYPERTENSIVE AGENTS |
| | ANTILIPIDEMIC AGENTS |
| | BILE ACID SEQUESTRANTS |
| | CHOLESTEROL ABSORPTION |
| | INHIBITORS & |
| | COMBINATIONS |
| | FIBRIC ACID DERIVATIVES |
| | HMG-CoA REDUCTASE |
| | INHIBITORS & |
| | COMBINATIONS |
| | MISCELLANEOUS |
| | ANTILIPIDEMIC AGENTS |
| | NICOTINIC ACID AGENTS |
| | BETA ADRENERGIC BLOCKING AGENTS |
| | BETA ADRENERGIC BLOCKING AGENTS |
| | WITH DIURETICS |
| | CALCIUM CHANNEL BLOCKERS & |
| | COMBINATIONS |
| | DIURETICS |
| | COMBINATION DIURETICS |
| | LOOP DIURETICS |
| | POTASSIUM-SPARING |
| | DIURETICS |
| | THIAZIDES & RELATED |
| | DIURETICS |
| | ENDOTHELIN RECEPTOR ANTAGONIST |
| | INOTROPIC AGENTS |
| | MISCELLANEOUS CARDIOVASCULAR |
| | AGENTS |
| | VASODILATORS |
| | CORONARY VASODILATORS |
| | NATRIURETIC PEPTIDES |
| | PULMONARY |
| | VASODILATORS |
| | VASOPRESSORS |
| | VASOPROTECTIVE AGENTS |
| CENTRAL NERVOUS SYSTEM | |
| DEPRESSANT | |
| CENTRAL NERVOUS SYSTEM | AMPHETAMINES |
| STIMULANTS | APPETITE SUPPRESSANTS |
| | MISCELLANEOUS CENTRAL NERVOUS |
| | SYSTEM STIMULANTS |
| CHOLINESTERASE | |
| INHIBITORS | |
| CONTRACEPTIVES | DEVICES |
| | INJECTABLE CONTRACEPTIVES |
| | ORAL CONTRACEPTIVES |
| | TRANSDERMAL CONTRACEPTIVES |
| CYSTIC FIBROSIS | |
| MANAGEMENT | |

D

| | |
|---|---|
| DIAGNOSTICS | ACTH TEST |
| | BRONCHIAL AIRWAY HYPERREACTIVITY |
| | GASTROINTESTINAL RADIOGRAPHY |
| | MYOCARDIAL PERFUSION |
| | SCINTIGRAPHY ADJUNCT |
| | PROSTATE CANCER |
| | RENAL FUNCTION TEST |
| | THYROID FUNCTION TEST |
| | THYROID RELEASING FACTOR |
| DIETARY SUPPLEMENTS | AMINO ACIDS & COMBINATIONS |
| | BLOOD MODIFIERS |
| | IRON & COMBINATIONS |
| | DIGESTIVE AIDS |
| | FIBER SUPPLEMENTS |
| | HERBAL COMBINATIONS |
| | MISCELLANEOUS HERBAL |
| | COMBINATIONS |
| | IMMUNE SYSTEM SUPPORT |
| | MINERALS & ELECTROLYTES |
| | CALCIUM & COMBINATIONS |
| | MAGNESIUM & |
| | COMBINATIONS |
| | MULTIMINERALS & |
| | COMBINATIONS |
| | PHOSPHOROUS & |
| | COMBINATIONS |
| | POTASSIUM & |
| | COMBINATIONS |
| | ZINC & COMBINATIONS |
| | MISCELLANEOUS DIETARY SUPPLEMENTS |
| | PRENATAL FORMULATIONS |
| | VITAMINS & COMBINATIONS |
| | GERIATRIC FORMULATIONS |
| | MULTIVITAMINS & |
| | COMBINATIONS |
| | MULTIVITAMINS WITH |
| | MINERALS |
| | PRENATAL FORMULATIONS |
| | THERAPEUTIC |
| | FORMULATIONS |
| | VITAMIN A & |
| | COMBINATIONS |
| | B VITAMINS & |
| | COMBINATIONS |
| | VITAMIN C & COMBINATIONS |
| | VITAMIN D ANALOGUES & |
| | COMBINATIONS |
| | VITAMIN E & COMBINATIONS |
| DOPAMINE RECEPTOR | |
| AGONISTS | |

E

EMERGENCY KITS
ENDOMETRIOSIS
MANAGEMENT
ENZYMES
ERECTILE DYSFUNCTION
THERAPY

F

FARRY DISEASE
MANAGEMENT
FERTILITY AGENTS
FOOT CARE PRODUCTS

G

| | |
|---|---|
| GASTROINTESTINAL | ANTACIDS |
| AGENTS | COMBINATION ANTACIDS |
| | MISCELLANEOUS ANTACID |
| | PREPARATIONS |
| | ANTIDIARRHEALS |
| | ANTIEMETICS |
| | ANTIFLATULENTS |
| | ANTI-INFLAMMATORY AGENTS |
| | ANTISPASMODICS & ANTICHOLINERGICS |
| | BOWEL EVACUANTS |
| | CYTOPROTECTIVE AGENTS |
| | DIGESTIVE ENZYMES |
| | DUODENAL ULCER ADHERENT COMPLEX |
| | HISTAMINE (H2) RECEPTOR ANTAGONISTS |
| | LAXATIVES |
| | BULK PRODUCING |
| | LAXATIVES |
| | EMOLLIENT LAXATIVES |
| | ENEMAS |
| | MISCELLANEOUS LAXATIVES |
| | SALINE LAXATIVES |
| | STIMULANT LAXATIVES & |
| | COMBINATIONS |
| | MISCELLANEOUS |
| | GASTROINTESTINAL AGENTS |
| | PROTON PUMP INHIBITORS |
| GAUCHER'S DISEASE | |
| MANAGEMENT | |
| GOUT PREPARATIONS | NONSTEROIDAL ANTI-INFLAMMATORY |
| | DRUGS (NSAIDS) |

H

| | |
|---|---|
| HOMEOPATHIC REMEDIES | ANTI-INFLAMMATORY AGENTS |
| | MISCELLANEOUS HOMEOPATHIC REMEDIES |
| | PAIN RELIEVERS |
| HORMONES | ANDROGEN & ESTROGEN COMBINATIONS |
| | ANDROGENS |
| | CALCITONIN |
| | ESTROGENS & COMBINATIONS |
| | GLUCOCORTICOIDS |
| | GLUCOSE ELEVATING AGENTS |
| | GONADOTROPIN INHIBITORS |
| | GONADOTROPIN RELEASING HORMONES |
| | (GNRH) |
| | GONADOTROPIN RELEASING |
| | HORMONES (GNRH) |
| | ANALOGUES |
| | GONADOTROPINS |
| | MENOTROPINS |
| | UROFOLLITROPINS |
| | GROWTH FACTOR |
| | GROWTH HORMONE |
| | GROWTH HORMONE RECEPTOR |
| | ANTAGONIST |
| | PROGESTIN & ESTROGEN COMBINATIONS |
| | PROGESTINS & COMBINATIONS |
| | SOMATOSTATIN ANALOGUES |
| | THYROID PREPARATIONS |
| | SYNTHETIC COMBINATIONS |
| | T3 & T4 |
| | SYNTHETIC T3 |
| | SYNTHETIC T4 |
| | VASOPRESSIN & DERIVATIVES |
| HYPERCALCEMIA | |
| MANAGEMENT | |
| HYPOCALCEMIA | |
| MANAGEMENT | |
| HYPONATREMIA | |
| MANAGEMENT | |

I

IMMUNOMODULATORS
IMMUNOSUPRESSIVES

M

| | |
|---|---|
| MEDICAL FOODS | |
| MIGRAINE PREPARATIONS | ERGOT DERIVATIVES & COMBINATIONS |
| | MISCELLANEOUS MIGRAINE PREPARATIONS |
| | SEROTONIN (5-HT) RECEPTOR AGONISTS |
| MOTION SICKNESS | |
| PRODUCTS | |
| MULTIPLE SCLEROSIS | |
| MANAGEMENT | |
| MUSCLE RELAXANTS | ACETYLCHOLINE INHIBITORS |
| | MISCELLANEOUS MUSCLE RELAXANTS |
| | NEUROMUSCULAR BLOCKING AGENTS |
| | SKELETAL MUSCLE RELAXANTS & |
| | COMBINATIONS |

N

| | |
|---|---|
| NASAL PREPARATIONS | ANTIBIOTICS & COMBINATIONS |
| | ANTICHOLINERGICS |
| | ANTI-INFLAMMATORY AGENTS |
| | STEROIDAL ANTI- |
| | INFLAMMATORY AGENTS |
| | HORMONES |
| | MISCELLANEOUS NASAL PREPARATIONS |
| | SYMPATHOMIMETICS & COMBINATIONS |
| NUCLEOSIDE ANALOGUES & | |
| COMBINATIONS | |
| NUCLEOTIDE ANALOGUES | |
| AND COMBINATIONS | |

O

| | |
|---|---|
| OBESITY MANAGEMENT | APPETITE SUPPRESSANTS |
| | LIPASE INHIIBITORS |
| OPHTHALMIC | ACETYLCHOLINE BLOCKING AGENTS |
| PREPARATIONS | ANTIHISTAMINE & MAST CELL STABILIZER |
| | COMBINATIONS |
| | ANTI-INFECTIVES |
| | ANTIBIOTICS & |
| | COMBINATIONS |
| | QUINOLONES |
| | SULFONAMIDES & |
| | COMBINATIONS |
| | ANTI-INFLAMMATORY AGENTS |
| | NONSTEROIDAL ANTI- |
| | INFLAMMATORY DRUGS |
| | (NSAIDS) |
| | STEROIDAL ANTI- |
| | INFLAMMATORY AGENTS & |
| | COMBINATIONS |
| | ARTIFICIAL TEARS/LUBRICANTS & |
| | COMBINATIONS |
| | BETA ADRENERGIC BLOCKING AGENTS |
| | BETA ADRENERGIC BLOCKING AGENT & |
| | CARBONIC ANHYDRASE |
| | INHIBITOR COMBINATIONS |
| | CARBONIC ANHYDRASE INHIBITORS |
| | MAST CELL STABILIZERS |
| | MISCELLANEOUS OPHTHALMIC |
| | PREPARATIONS |
| | PHOTODYNAMIC THERAPY AGENTS |
| | PROSTAGLANDINS |
| | SYMPATHOMIMETICS & COMBINATIONS |
| | VASOCONSTRICTORS |
| | VITAMINS & COMBINATIONS |
| OSTEOPOROSIS | BISPHOSPHONATES & COMBINATIONS |
| PREPARATIONS | HORMONAL AGENTS |
| | CALCITONIN |
| | ESTROGENS & |
| | COMBINATIONS |
| | MISCELLANEOUS |
| | HORMONAL AGENTS |
| | SELECTIVE ESTROGEN |
| | RECEPTOR MODULATORS |
| OTIC PREPARATIONS | ANTIBIOTIC & STEROID COMBINATIONS |

P

| | |
|---|---|
| PATENT DUCTUS | |
| ARTERIOSUS AGENTS | |
| PHOSPHATE BINDERS | |
| PORPHYRIA AGENTS | |
| PROSTAGLANDINS | |
| PYSCHOTHERAPEUTIC | ANTIANXIETY AGENTS |
| AGENTS | BENZODIAZEPINES & |
| | COMBINATIONS |
| | MISCELLANEOUS |
| | ANTIANXIETY AGENTS |
| | ANTIDEPRESSANTS |
| | MISCELLANEOUS |
| | ANTIDEPRESSANTS |
| | MONOAMINE OXIDASE |
| | INHIBITORS (MAOI) |
| | SELECTIVE SEROTONIN |
| | REUPTAKE INHIBITORS (SSRI) |
| | TRICYCLIC |
| | ANTIDEPRESSANTS & |
| | COMBINATIONS |
| | ANTIPANIC AGENTS |
| | ANTIPSYCHOTIC AGENTS |
| | MISCELLANEOUS |
| | ANTIPSYCHOTIC AGENTS |
| | PHENOTHIAZINES & |
| | COMBINATIONS |
| | BIPOLAR AGENTS |
| | OBSESSIVE-COMPULSIVE DISORDER |
| | MANAGEMENT |
| | SELECTIVE SEROTONIN |
| | REUPTAKE INHIBITORS (SSRI) |

R

| | |
|---|---|
| RESINS, ION EXCHANGE | |
| RESPIRATORY AGENTS | ANTI-INFLAMMATORY AGENTS |
| | STEROIDAL ANTI- |
| | INFLAMMATORY AGENTS & |
| | COMBINATIONS |
| | ANTITUSSIVES |
| | NARCOTIC ANTIITUSSIVES & |
| | COMBINATIONS |
| | NON-NARCOTIC |
| | ANTITUSSIVES & |
| | COMBINATIONS |
| | BRONCHODILATORS |
| | ANTICHOLINERGICS |
| | ANTICHOLINERGICS WITH |
| | SYMPATHOMIMETICS |
| | SYMPATHOMIMETICS & |
| | COMBINATIONS |
| | XANTHINE DERIVATIVES & |
| | COMBINATIONS |
| | DECONGESTANTS & COMBINATIONS |
| | DECONGESTANTS, EXPECTORANTS & |
| | COMBINATIONS |
| | DEVICES |
| | ENZYMES |
| | EXPECTORANTS & COMBINATIONS |
| | LEUKOTRIENE ANTAGONISTS |
| | LEUKOTRIENE FORMATION INHIBITORS |
| | LUNG SURFACTANTS |
| | MISCELLANEOUS COLD & COUGH |
| | PRODUCTS WITH ANALGESICS |
| | MISCELLANEOUS RESPIRATORY AGENTS |

S

| | |
|---|---|
| SEDATIVES & HYPNOTICS | BARBITURATES |
| | BENZODIAZEPINES |
| | MISCELLANEOUS SEDATIVES & HYPNOTICS |
| SKIN & MUCOUS MEMBRANE | ACNE PREPARATIONS |
| AGENTS | ANALGESICS & COMBINATIONS |
| | ANESTHETICS & COMBINATIONS |
| | ANORECTAL PREPARATIONS |
| | ANTIHISTAMINES & COMBINATIONS |
| | ANTI-INFECTIVES |
| | ANTIBIOTICS & |
| | COMBINATIONS |
| | ANTIFUNGALS & |
| | COMBINATIONS |
| | ANTIVIRALS |
| | MISCELLANEOUS ANTI- |
| | INFECTIVES & |
| | COMBINATIONS |
| | ANTINEOPLASTICS |
| | ANTIPERSPIRANTS |
| | ANTIPRURITICS |
| | ANTIPSORIATIC AGENTS |
| | BURN PREPARATIONS |
| | DEODORANTS |
| | DRYING AGENTS |
| | EMOLLIENTS & MOISTURIZERS |
| | ENZYMES & COMBINATIONS |
| | HAIR GROWTH STIMULANTS |
| | KERATOLYTICS |
| | MISCELLANEOUS SKIN & MUCOUS |
| | MEMBRANE AGENTS |
| | MOUTH & THROAT PRODUCTS |
| | CANKER SORE |
| | PREPARATIONS |
| | DENTAL PREPARATIONS |
| | LOZENGES & SPRAYS |
| | MISCELLANEOUS MOUTH & |
| | THROAT PRODUCTS |
| | ORAL RINSES |
| | SALIVA PRODUCTS |
| | PHOTOSENSITIZING AGENTS |
| | SCAR TISSUE TREATMENT |
| | SHAMPOOS |
| | STEROIDS & COMBINATIONS |
| | WART PREPARATIONS |
| | WOUND CARE PRODUCTS |
| SMOKING CESSATION AIDS | |

U

| | |
|---|---|
| URINARY TRACT AGENTS | ACIDIFIERS |
| | ALKALINIZERS |
| | ANALGESICS & COMBINATIONS |
| | ANTIBACTERIALS |
| | ANTISPASMODICS |
| | BENIGN PROSTATIC HYPERPLASIA (BPH) |
| | THERAPY |
| | CALCIUM OXALATE STONE PREVENTION |
| | CYTOPROTECTIVE AGENTS |
| | IMPOTENCE AGENTS |
| | MISCELLANEOUS URINARY TRACT AGENTS |

V

| | |
|---|---|
| VAGINAL PREPARATIONS | ANTI-INFECTIVES |
| | MISCELLANEOUS ANTI- |
| | INFECTIVES & |
| | COMBINATIONS |
| | ESTROGENS |
| | PROSTAGLANDINS |
| VASODILATORS | CEREBRAL VASODILATORS |
| VITAMINS | |

Examples of medicating active ingredients contemplated for use in the present invention include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin (available as Bayer®), acetaminophen, and combinations thereof that may optionally include caffeine.

Other active ingredients that may be used in the present invention include anti-diarrheals such as Imodium AD®, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Specific active ingredients contemplated for use herein include adrenergic agonists such as clonidine; anxiolytics such as alprazolam (available as Xanax®); anti-psychotics such as clozapine (available as Clozaril®) and haloperidol (available as Haldol®); non-steroidal anti-inflammatories (NSAID's) such as dicyclofenac (available as Voltaren®) and etodolac (available as Lodine®), anti-histamines such as loratadine (available as Claritin®), astemizole (available as Hismanal®), nabumetone (available as Relafen®), fexofenadine (available as Allegra®), and clemastine (available as Tavist®); anti-emetics such as granisetron hydrochloride (available as Kytril®), serotonin 5-HT3 receptor antagonists such as ondansetron (available as Zofran®) and nabilone (available as Cesamet™); bronchodilators such as salbutamol (aka albuterol, available as Ventolin®), albuterol sulfate (available as Proventil®); anti-depressants such as fluoxetine hydrochloride (available as Prozac®), sertraline hydrochloride (available as Zoloft®), and paroxetine hydrochloride (available as Paxil®); anti-migraines such as sumatriptan (available as Imigran®), ACE-inhibitors such as enalapril (available as Vasotec®), captopril (available as Capoten®) and lisinopril (available as Prinivil® and Zestril®); anti-Alzheimer's agents, such as nicergoline; calcium channel blocker (CCB) such as nifedipine (available as Procardia® and Adalat®), and verapamil hydrochloride (available as Calan®); opioid analgesics such as fentanyl (available as Sublimaze®), alfentanil, sufentanil, remifentanil, carfentanil, and lofentanil; cough suppressants such as dextromethorphan; local anesthetics such as benzocaine (available as Cepacol® and Anbesol®); peptide hormones such as insulin; oral contraceptives such as estrogen (estradiol) and a progestogen (progestin); vaccines such as killed vaccines (e.g., influenza vaccine, cholera vaccine, bubonic plague vaccine, polio vaccine, hepatitis A vaccine, and rabies vaccine); attenuated vaccines (e.g., yellow fever, measles, rubella, and mumps); toxoid vaccines (e.g., tetanus and diphtheria); subunit vaccines (e.g., subunit vaccine against Hepatitis B virus, virus-like particle (VLP) vaccine against human papillomavirus, and the hemagglutinin and neuraminidase subunits of the influenza virus); fluoridating agents such as sodium fluoride, sodium monofluorophosphate (MFP) and stannous fluoride; stimulants such as caffeine, theobromine, theophylline, yohimbine, and nicotine; energy boosters such as methylxanthines (e.g., caffeine), B vitamins (e.g., Vitamin B12), herbs, guarana, yerba mate, acai, taurine, various forms of ginseng, maltodextrin, inositol, carnitine, creatine, glucuronolactone, ginkgo biloba, bitter orange extract, coenzyme Q10, amino acids (e.g., L-carnitine), bee pollen, royal jelly, green tea extract, spirulina, gotu kola, and glucose; opioid antidiarrheals such as loperamide (available as Imodium®); sports supplements such as fish oil, dietary protein, creatine, caffeine, glutamine, essential fatty acids (e.g., (alpha-linolenic acid and linoleic acid), prohormones (e.g., chrysin and 4-androstene-3,6,17-trione), and testosterone boosters (e.g., Fenugreek, Eurycoma longifolia, D-Aspartic acid, Boron, L-Carnitine and Tribulus terrestris); analgesics such as non-steroidal anti-inflammatory drugs (NSAIDs); COX-2 inhibitors such as rofecoxib, celecoxib and etoricoxib; opiates such as morphine, diacetylmorphine, codeine, oxycodone, hydrocodone, dihydromorphine, pentazocine, butorphanol, and pethidine; dietary supplements such as melatonin (N-acetyl-5-methoxytryptamine), vitamins, minerals, fiber, fatty acids, and amino acids; electrolytes such as sodium (NO, potassium (K⁺), calcium (Ca²⁺), magnesium (Mg²⁺), chloride (Cl⁻), hydrogen phosphate (HPO₄²⁻), and hydrogen carbonate (HCO₃⁻); artificial sweeteners and natural sweeteners.

Erectile dysfunction therapies include, e.g., drugs for facilitating blood flow to the penis, and for effecting autonomic nervous activities, such as increasing parasympathetic (cholinergic) and decreasing sympathetic (adrenergic) activities. Useful non-limiting drugs include sildenafil and pharmaceutically acceptable salts thereof, such as Viagra®, tadalafil, such as Cialis®, vardenafil and pharmaceutically acceptable salts thereof, such as Levitra®, apomorphine and pharmaceutically acceptable salts thereof, such as Uprima®, yohimbine hydrochloride such as Aphrodyne®, and alprostadil such as Caverject®.

The popular H₂-antagonists which are contemplated for use in the present invention include cimetidine, ranitidine hydrochloride, famotidine, nizatidine, ebrotidine, mifentidine, roxatidine, pisatidine, and aceroxatidine.

Specific suitable active ingredients include ondansetron (available as Zuplenz® and Zofran®), diphenhydramine (available as Benadryl®); simethicone (available as Gas-X®); melatonin (available as Melatonin PM®); benzocaine (available as Orajel®); buprenorphine and naloxone (available as Suboxone®); buprenorphine (available as Subutex®); phenylephrine or pseudoephedrine (available as Sudafed®); acetaminophen, chlorpheniramine maleate, dextromethorphan hydrobromide, and pseudoephedrine hydrochloride (available as Theraflu®); and paracetamol and phenylephrine hydrochloride (available as Lemsip®).

Additional specific active ingredients employed in the present invention may include allergens or antigens, such as, but not limited to, plant pollens from grasses, trees, or ragweed; animal danders, which are tiny scales shed from the skin and hair of cats and other furred animals; and insects, such as house dust mites, bees, and wasps.

In specific embodiments, the thin film can be used to deliver poorly water soluble (and/or poorly bioavailable) active ingredients. In some embodiments, the administration can be via at least one of a buccal and sublingual administration. Such an administration can take advantage of the higher systemic absorption of many active ingredients via a buccal or sublingual administration, compared to the oral route (e.g., use of a solid oral dosage or liquid form). In other embodiments, the administration can be topical, to a wound. In other embodiments, the administration can be ocular or nasal. In other embodiments, the administration can be intravaginal.

The poorly soluble active ingredient used herein is an active ingredient of which solubility in water is extremely low and of which absorbability is inferior in normal oral administration, and is referred to, for example, as the active ingredient defined as “nearly insoluble” or “extremely difficult to be dissolved” in U.S. Pharmacopoeia. In U.S. Pharmacopoeia, the solubility of an active ingredient can be defined as the degree of dissolvability within 30 minutes when the active ingredient is placed in a solvent and shaken for 30 seconds every 5 minutes at 20±5° C. after making powder in the case of the active ingredient being solid. “Nearly insoluble” is referred to the characteristic where an amount of 10,000 ml or more of the solvent is required to dissolve 1 g or 1 ml of the active ingredient. “Extremely difficult to be dissolved” is referred to the characteristic where a solvent amount of 1,000 to 10,000 ml is required to dissolve 1 g or 1 ml of the active ingredient. Specifically, such active ingredients include, e.g., azelastine (available as Optivar®), betamethasone (available as Celestone®), bifonazole (available as Cansespor®), calcipotriol (available as Dovonex®), calcitriol (available as Rocaltrol®), clobetasol propionate (available as Cormax®), clotrimazole (available as Mycelex®), chloramphenicol (available as Chloromycetin®), clocortolone (available as Cloderm®), diazepam (available as Valium®), digitoxin (available as Digitaline®), digoxin (available as Lanoxin®), dexametasone (available as Dexacort®), fluconazole (available as Diflucan®), fluticasone propionate (available as Flonase®), fluticasone furoate (available as Veramyst®), griseofulvin (available as Grifulvin®), itraconazole (available as Sporanox®), methylprednisolone (available as Medrol®), mometasone furoate (available as Nasonex®), naproxen (available as Aleve®), nilvadipine, nifedipine (available as Adalat®), prednicarbate (available as Dermatop®), prednisone (available as Deltasone®), phenytoin (available as Dilantin®), pimecrolimus (available as Elidel®), tacalcitol (1,24-dihydroxyvitamin D₃), tacrolimus (available as Protopic®), and terbinafine (available as Lamisil®).

In specific embodiments, the oral thin film can be used to deliver various active ingredients that typically degrade in the gastrointestinal (GI) tract, due, e.g., to stomach acid, bile, digestive enzymes, and other first-pass effects. In additional specific embodiments, the oral thin film can be used to deliver various active ingredients that typically have an irritant effect on the stomach (e.g., aspirin, esomeprazole, and omeprazole). In additional specific embodiments, the oral thin film can be used to deliver various active ingredients that typically have been known to cause a fishy reflux (e.g., fish oil (omega-3 fatty acids)). In specific embodiments, these active ingredients can be delivered via at least one of a buccal and sublingual administration. In alternative specific embodiments, these active ingredients can be delivered orally, wherein the active ingredients are coated or encapsulated, such that the coating or encapsulation is stable at the highly acidic pH found in the stomach (e.g., pH ˜3), but breaks down rapidly at a less acidic pH (e.g., pH 7-9) environment present in the small intestine. As such, any drug typically enterically coated (e.g., diclofenac sodium, naprosyn, and aspirin) can be employed into the manufacturing of the thin film described herein.

### Combination Therapy

Active ingredients described herein can also be used in combination with other active ingredients. Such combinations are selected based on the condition to be treated, individual reactivities of the ingredients, cross-reactivities of ingredients, and pharmaco-properties of the combination.

It is also possible to combine any active ingredient described herein with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide “synergy” and “synergistic effect”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the active ingredients are administered or delivered sequentially, e.g., in separate thin films. In general, during alternation therapy, an effective dosage of each active ingredient described herein is administered sequentially, i.e. serially, whereas in combination therapy, effective dosages of two or more active ingredients described herein are administered together.

Each of the active ingredients in the combination may be effective in treating the same disease or disorder (e.g., each active ingredient may be an NSAID, effective in treating inflammation). In contrast, the combination may include active ingredients effective in treating different diseases or disorders (e.g., one active ingredient may be an NSAID, effective in treating inflammation, and another active ingredient may be an analgesic, effective in treating pain). As such, the combination may include active ingredients each having the same (or different) therapeutic use or mechanism of action from each of the other active ingredients in the combination.

The combination of active pharmaceutical ingredients can be particularly effective in the treatment of psychiatric patients (depression and psychosis in particular), hospice care, antibiotic therapies, treatment of hypertension, and hormone replacement therapies. The combination therapies can reduce “pill burden” for the named classes of patients (including hospice). OTF combination therapies can reduce, eliminate or minimize the occurrence or likelihood of “cheeking”, a practice employed by patients (e.g., psychiatric patients) that allows them to avoid compliance with prescribed medication regimens.

### Hospice

The following combinations of active ingredients may be particularly useful in the treatment of hospice patients:

Lorazepam, diphenhydramine, haloperidol, and metoclopramide;

Lorazepam, diphenhydramine, and haloperidol;

Lorazepam, diphenhydramine, and metoclopramide;

Diphenhydramine, dexamethasone, and metoclopramide;

Lorazepam and diphenhydramine;

Lorazepam, haloperidol, and metoclopramide;

Diphenhydramine, haloperidol, and metoclopramide;

Promethazine and metoclopramide;

Promethazine, metoclopramide, and lorazepam; or

Haloperidol and lorazepam.

The table below illustrates additional combinations of active ingredients that may be particularly useful in the treatment of hospice patients.

| Weight (mg) | API |
|---|---|
| 0.5 | lorazepam |
| 12.5 | diphenhydramine |
| 0.5 | haloperidol |
| 10.0 | metoclopramide |
| 0.5 | lorazepam |
| 12.5 | diphenhydramine |
| 0.5 | haloperidol |
| 0.5 | lorazepam |
| 12.5 | diphenhydramine |
| 10.0 | metoclopramide |
| 20.0 | diphenhydramine |
| 4.0 | dexamethasone |
| 4.0 | metoclopramide |
| 1.0 | lorazepam |
| 50.0 | diphenhydramine |
| 0.5 | lorazepam |
| 0.5 | haloperidol |
| 5.0 | metoclopramide |
| 12.5 | diphenhydramine |
| 0.5 | haloperidol |
| 5.0 | metoclopramide |
| 25.0 | promethazine |
| 10.0 | metoclopramide |
| 25.0 | promethazine |
| 10.0 | metoclopramide |
| 1.0 | lorazepam |
| 0.5 | haloperidol |
| 0.5 | lorazepam |

### Geriatric, Pediatric, and Psychiatric (Specific APIs)

The table below illustrates additional combinations of active ingredients that may be particularly useful in the treatment of geriatric (GER), pediatric (PED), and/or psychiatric (PSY) patients.

| | | Class | |
|---|---|---|---|
| | | (Geriatric, | |
| | Weights | Pediatric, | API |
| Combination Name | (mg) | Psychiatric) | Information |
| Levoamphetamine | 10, 15, | Ger | 3:1 D/L mixture |
| Dextroamphetamine | 20, 30 | Ped | of amphetamine |
| | | | enantiomers. |
| | | | Depression |
| Spironolactone | 25 | Ger | Hypertension |
| Hydrochlorothiazide | 25 | | |
| diphenoxylate | 2.5 | Ger | Diarrhea |
| hydrochloride | 0.025 | Ped | |
| atropine sulfate | | | |
| hydrochlorothiazide | 25 | Ger | Hypertension |
| triamterene | 37.5 | | |
| Olanzapine | 3 | Psy | Bipolar I Disorder, |
| fluoxetine | 25 | | Treatment Resistant |
| | | | Depression |
| Estriol | variable | Perimen- | Hormone replacement |
| Estrone | | opausal, | therapy |
| Estradiol | | Menopausal | |
| Terazosin | variable | Ger | Benign Prostatic |
| Finasteride | | | Hypertrophy (BPH) |

### Geriatric, Pediatric, and Psychiatric (Therapeutic Class of APIs)

The table below illustrates additional combinations of active ingredients (by therapeutic class), that may be particularly useful in the treatment of geriatric (GER), pediatric (PED), and/or psychiatric (PSY) patients.

| | Class | |
|---|---|---|
| Combination | (Geriatric, Pediatric, | |
| Therapeutic Class | Psychiatric) | Treatment |
| Atypical | Psy | Depression |
| Antidepressants | | |
| Mixed Classes of | Psy | Depression |
| Antipsychotics | | |
| Lithium + MAOI, | Psy | Depression |
| TCA, SSRI, or SNRI | | |
| L-Triiodothyronine + | Psy | Depression |
| TCA, or MAOI | | |
| Mixed Classes of | Psy | Depression |
| Stimulants (such as | | |
| Dexadrine or | | |
| Methylphenidate + | | |
| other stimulants) | | |
| Mixed Classes of | All | Hypertension |
| Antihypertensives | | |
| Antihypertensives + | All | Hypertension |
| Diuretics | | |
| Angiotensin II | All | Hypertension |
| Receptor Antagonist + | | |
| Diuretic | | |
| Calcium Channel | All | Hypertension |
| Blocker + ACE | | |
| Inhibitor | | |
| ACE Inhibitor + | All | Chronic Kidney |
| Angiotensin II | | Disease |
| Receptor Antagonist | | |
| Diuretic + Beta Blocker + | All | Diabetes |
| ACE Inhibitor + | | |
| Angiotensin II | | |
| Receptor Antagonist + | | |
| Calcium Channel | | |
| Blocker | | |
| Diuretic + Beta Blocker + | All | Heart Failure |
| ACE Inhibitor + | | |
| Angiotensin II | | |
| Receptor Antagonist + | | |
| Aldosterone Antagonist | | |
| Diuretic + Beta Blocker + | All | High Coronary |
| ACE Inhibitor + | | Disease Risk |
| Calcium Channel | | |
| Blocker | | |
| Beta Blocker + ACE | All | Post myocardial |
| Inhibitor + Aldosterone | | infarction |
| Antagonist | | |
| Diuretic + ACE | All | Recurrent stroke |
| Inhibitor | | prevention |
| Beta blocker + Diuretic | All | Hypertension |
| Vasodilator + Diuretic | All | Hypertension |
| Diuretic + Diuretic | All | Hypertension |
| Alpha Blockers and 5- | Ger | BPH |
| Alpha-Reductase | | |
| Inhibitors | | |
| Anticholinergic and | Ger | BPH |
| Alpha Blocker | | |
| Sertraline + stimulant | Psy | Depression + |
| | | Anxiety |

TCA = Tricyclic Antidepressant
SSRI = Selective Serotonin Reuptake Inhibitor
SNRI = Serotonin-Norepinephrine Reuptake Inhibitor
MAOI = Monoamine Oxidase Inhibitor

### Additional/Optional Components

A variety of optional components and fillers also may be added to the films. These may include, without limitation: surfactants; plasticizers; polyalcohols; anti-foaming agents, such as silicone-containing compounds, which promote a smoother film surface by releasing oxygen from the film; thermo-setting gels such as pectin, carrageenan, and gelatin, which help in maintaining the dispersion of components; inclusion compounds, such as cyclodextrins and caged molecules; coloring agents; and flavors. In some embodiments, more than one active ingredient may be included in the film.

Additives may be included in the films. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active agent(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids, polyacrylamides, and polyacrylic acid esters, polymethacrylic acids, polymethacrylamides, and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers. Inventive films may further include compounds such as butylated hydroxytoluene.

Further additives include inorganic fillers, such as the oxides of magnesium aluminum, silicon, titanium, etc. desirably in a concentration range of about 0.02% to about 3% by weight and desirably about 0.02% to about 1% based on the weight of all film components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

There may further be added compounds to improve the texture and/or flow properties of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e., with C₁₂-, C₁₄-, C₁₆-, C₁₈-, C₂₀- and C₂₂-fatty acids.

The total amounts used of the fats, mono-, di-glycerides and/or lecithins may be up to about 5% and preferably within the range of about 0.5% to about 2% by weight of the total film composition.

It further may be useful to add silicon dioxide, calcium silicate, or titanium dioxide in a concentration of about 0.02% to about 1% by weight of the total composition. These compounds typically act as flow agents.

Other optional ingredients include binders which contribute to the ease of formation and general quality of the films. Non-limiting examples of binders include starches, pregelatinized starches, gelatin, polyvinylpyrrolidone, methylcellulose, sodium carboxymethylcellulose, ethylcellulose, polyacrylamides, polyvinyloxoazolidone, and polyvinylalcohols. If desired, the film may include other additives, such as keratin, or proteins, including proteins that are useful in forming a gel, such as gelatin.

Further potential additives include solubility enhancing agents, such as substances that form inclusion compounds with active ingredients. Such agents may be useful in improving the properties of very insoluble and/or unstable actives. In general, these substances are doughnut-shaped molecules with hydrophobic internal cavities and hydrophilic exteriors. Insoluble and/or instable actives may fit within the hydrophobic cavity, thereby producing an inclusion complex, which is soluble in water. Accordingly, the formation of the inclusion complex permits very insoluble and/or instable actives to be dissolved in water. A particularly desirable example of such agents are cyclodextrins, which are cyclic carbohydrates derived from starch. Other similar substances, however, are considered well within the scope of the present invention.

### Kits

Pharmaceutical kits are also within the ambit of the present invention. Such kits include a therapeutically effective amount of a thin film as described herein. Sterilization of the thin film and/or packaging material may be carried out using conventional sterilization methodology well-known to those skilled in the art. Instructions or printed indicia, either as inserts or as labels, indicating quantities of the components to be administered, guidelines for administration, may also be included in the kit.

### Utility

The thin films described herein can be useful to deliver a high load of active ingredients to the intended target. The thin film can be placed, e.g., in the mouth thereby administering active ingredients via absorption in the mouth (buccally or sublingually), the stomach (gastrically), and/or via the small intestines (enterically). Such an OTF will be edible (suitable for human consumption), and pharmaceutically acceptable. The thin films can be prepared typically using hydrophilic polymers that rapidly dissolve on the tongue or buccal cavity, delivering the active ingredient to the systemic circulation via dissolution when contact with liquid is made. The thin film can also be used to adhere to mucosal tissue (e.g., at least one of mouth, nose, eye, vagina, and rectum), thereby locally delivering the active ingredient(s) to those bodily tissues. As such, the mucoadhesive films may be used for the administration of an active to any of several body surfaces, especially those including mucous membranes, such as oral, rectal, vaginal, opthalmological, the surface of a wound, either on a skin surface or within a body such as during surgery, and similar surfaces.

The thin film can be manufactured to include a relatively high load (e.g., up to about 40 wt. %) of active ingredient. The active ingredient can include active pharmaceutical ingredients (APIs) (e.g., prescription (Rx), over the counter (OTC), and biologicals), veterinary agents, vitamins, nutraceuticals, supplements (e.g., dietary, nutritional, and herbal), and cosmetics. As such, the active ingredients described herein can be useful to treat a disease or disorder typically encountered by the target subject (e.g., human, animal, etc.). Selection of the appropriate active ingredient will influence each of the target subject (e.g., human, animal, etc.), as well as the disease or disorder to be treated. From a practical perspective, selection of both the target subject and the disease or disorder to be treated will influence the selection of the active ingredients that is employed. Furthermore, the ingredients used in construction of the films may be selected to allow for a range of disintegration times for the films.

The films may be applied under or to the tongue of the mammal. When this is desired, a specific film shape, corresponding to the shape of the tongue may be preferred. Therefore the film may be cut to a shape where the side of the film corresponding to the back of the tongue will be longer than the side corresponding to the front of the tongue. Specifically, the desired shape may be that of a triangle or trapezoid. Desirably, the film will adhere to the oral cavity preventing it from being ejected from the oral cavity and permitting more of the active to be introduced to the oral cavity as the film dissolves.

Another use for the thin films described herein takes advantage of the films' tendency to dissolve quickly when introduced to a liquid. An active ingredient may be introduced to a liquid (or liquid containing substance) by preparing a film as described herein, introducing it to a liquid (or liquid containing substance), and allowing it to dissolve. This may be used either to prepare a liquid dosage form of an active. This may also be used to flavor a beverage or food product, or to add at least one of a sweetener, electrolytes, nutrients, nutraceuticals, active ingredient, vitamins, and protein to a beverage or food product.

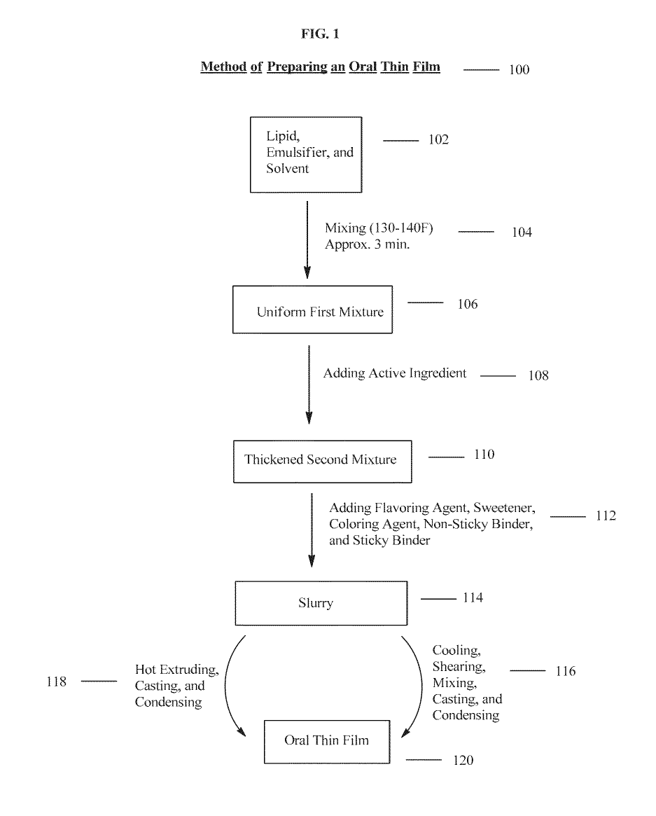

Referring to FIG. 1, an example of a block flow diagram illustrating a method of preparing an oral thin film (100) is shown, according to some embodiments. A combination of lipid, emulsifier, and solvent (102) is mixed to provide a uniform first mixture (106). Active ingredient is added (108) to the uniform first mixture (106), to form a thickened second mixture (110). Flavoring agent, sweetener, coloring agent, non-sticky binder, and sticky binder are added to the thickened second mixture (110), to provide a slurry (114). The slurry (114) is cooled, sheared, mixed, cast, and condensed (116) to provide an oral thin film (120). Alternatively, the slurry (114) is hot extruded, cast, and condensed (118) to provide an oral thin film (120).

The combination of lipid, emulsifier, and solvent (102) is mixed (104) to provide a uniform first mixture (106). The mixing (104) will typically be carried out at an elevated temperature (e.g., about 130-140° F. or 54.4-60° C.), sufficient to provide the uniform first mixture (106). Additionally, mixing (104) will typically be carried out for a suitable period of time (e.g., about 3 minutes), sufficient to provide the uniform first mixture (106). The mixing (104) can be carried out in any suitable manner, employing suitable equipment known to those of skill in the art, sufficient to provide the uniform first mixture (106). For example, the mixing (104) can include blending, which can be carried out employing a blender.

The uniform first mixture (106) and active ingredient can be contacted (108), to provide a thickened second mixture (110). Typically, the active ingredient can be added (108) to the uniform first mixture (106). The contacting (108) can include mixing. The mixing (108) will typically be carried out at room temperature (e.g., about 70° F.), and for a suitable period of time (e.g., up to about 5 minutes), sufficient to provide the thickened second mixture (110). The mixing (108) can be carried out in any suitable manner, employing suitable equipment known to those of skill in the art, sufficient to provide the thickened second mixture (110). For example, the mixing (108) can include blending, which can be carried out employing a blender.

Flavoring agent, sweetener, coloring agent, non-sticky binder, and sticky binder are contacted with the thickened second mixture (110), to provide a slurry (114). Typically, the flavoring agent, sweetener, coloring agent, non-sticky binder, and sticky binder can be added (112) to the thickened second mixture (110), to provide a slurry (114). The contacting can include mixing. The mixing will typically be carried out at room temperature (e.g., about 70° F.), and for a suitable period of time (e.g., up to about 5 minutes), sufficient to provide the slurry (114). The mixing can be carried out in any suitable manner, employing suitable equipment known to those of skill in the art, sufficient to provide the slurry (114). For example, the mixing can include blending, which can be carried out employing a blender.

The slurry (114) is hot extruded, cast, and condensed (118), to provide the thin film (120). With some active ingredients (e.g., caffeine), upon cooling, the active ingredient will fall out of solution (i.e., precipitate), due to poor solubility issues. With such active ingredients, the slurry (114) can be cooled, sheared, mixed, cast, and condensed (116) to provide the thin film (120).

The slurry (114) will typically have up to about 85 wt. % water. As such, the condensing can be carried out until the thin film (120) has the requisite amount of water (e.g., about 4-18 wt. %). Typically, the condensing will be carried out at an elevated temperature (e.g., at least about 70° C. (158° F.)). Additionally, the condensing will typically be carried out for an extended period of time (e.g., at least about 10 minutes). The condensing can be carried out in any suitable manner, employing suitable equipment known to those of skill in the art, sufficient to provide the thin film (120). For example, the condensing can be carried out employing an oven that utilizes passive convection. Alternatively, the condensing can be carried out employing an oven that utilizes active convection. In addition to (or in replace of) the elevated temperature, the condensing can be carried out at a reduced pressure (e.g., in vacuo).

Specific ranges, values, and embodiments provided below are for illustration purposes only and do not otherwise limit the scope of the invention, as defined by the claims. The specific ranges, values, and embodiments described below encompass all combinations and sub-combinations of each disclosed range, value, and embodiment, whether or not expressly described as such.

### Specific Ranges, Values, and Embodiments

In specific embodiments, the thin film is an oral thin film.

In specific embodiments, the thin film is an oral thin film, which is edible and pharmaceutically safe and effective.

In specific embodiments, the thin film is configured for application to mucosal tissue or a mucosal surface.

In specific embodiments, the thin film is configured for application to at least one of the mouth, buccal cavity, nose, eye, vagina, and rectum.

In specific embodiments, the thin film is configured for application to the mouth.

In specific embodiments, the thin film is configured for application to the buccal cavity.

In specific embodiments, the thin film is configured for application to the nose.

In specific embodiments, the thin film is configured for application to the eye.

In specific embodiments, the thin film is configured for application to the vagina.

In specific embodiments, the thin film is configured for application to the rectum.

In specific embodiments, the thin film is configured for delivering the active ingredient(s) to at least one of the mouth, buccal cavity, nose, eye, vagina, and rectum.

In specific embodiments, the thin film is configured for delivering the active ingredient(s) to the mouth.

In specific embodiments, the thin film is configured for delivering the active ingredient(s) to the buccal cavity.

In specific embodiments, the thin film is configured for delivering the active ingredient(s) to the nose.

In specific embodiments, the thin film is configured for delivering the active ingredient(s) to the eye.

In specific embodiments, the thin film is configured for delivering the active ingredient(s) to the vagina.

In specific embodiments, the thin film is configured for delivering the active ingredient(s) to the rectum.

In specific embodiments, the solvent includes at least one of water and ethanol.

In specific embodiments, the solvent includes water and ethanol.

In specific embodiments, the solvent includes water.

In specific embodiments, the solvent includes ethanol.

In specific embodiments, the solvent is present in at least about 2 wt. %.

In specific embodiments, the solvent is present in up to about 24 wt. %.

In specific embodiments, the solvent is present in about 2-24 wt. %.

In specific embodiments, the solvent is present in at least about 4 wt. %.

In specific embodiments, the solvent is present in up to 12 wt. %.

In specific embodiments, the solvent is present in about 4-12 wt. %.

In specific embodiments, the solvent is present in about 8 wt. %.

In specific embodiments, the binder includes at least one of pectin, microcrystalline cellulose, xanthan gum, locust bean gum, guar gum, gum arabic, gum tragacanth, gum karaya, beta glucan, glucomannan, tapioca starch, carrageenan, xanthan gum, gellan gum, alginic acid or sodium alginate, konjac gum, tara gum, chitosan, agar, maltodextrin, polyvinyl alcohol, pullulan, polycarbophil, povidone, hydroxypropyl methylcellulose, hydroxypropyl cellulose, hydroxypropyl starch, polyacrylic acid, and polyethylene glycol.

In specific embodiments, the binder is acidic.

In specific embodiments, the binder includes at least one of pectin and microcrystalline cellulose.

In specific embodiments, the binder includes pectin.

In specific embodiments, the binder includes microcrystalline cellulose.

In specific embodiments, the binder is sticky.

In specific embodiments, the binder is non-sticky.

In specific embodiments, the binder is present in at least about 4 wt. %.

In specific embodiments, the binder is present in up to about 50 wt. %.

In specific embodiments, the binder is present in about 4-50 wt. %.

In specific embodiments, the binder is present in at least about 10 wt. %.

In specific embodiments, the binder is present in up to about 36 wt. %

In specific embodiments, the binder is present in about 10-36 wt. %.

In specific embodiments, the binder is present in about 25 wt. %.

In specific embodiments, the lipid includes at least one of almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, walnut oil, and watermelon seed oil.

In specific embodiments, the lipid includes at least one of hydroxylated lecithin and deodorized cocoa butter.

In specific embodiments, the lipid includes hydroxylated lecithin.

In specific embodiments, the lipid includes deodorized cocoa butter.

In specific embodiments, the lipid is present in at least about 4 wt. %.

In specific embodiments, the lipid is present in up to about 22 wt. %.

In specific embodiments, the lipid is present in about 4-22 wt. %.

In specific embodiments, the lipid is present in at least about 10 wt. %.

In specific embodiments, the lipid is present in up to about 18 wt. %.

In specific embodiments, the lipid is present in about 10-18 wt. %.

In specific embodiments, the lipid is present in about 14.8 wt. %.

In specific embodiments, the emulsifier includes at least one of glycerin, propylene glycol, and polyethylene glycol.

In specific embodiments, the emulsifier includes glycerin.

In specific embodiments, the emulsifier is present in at least about 3 wt. %.

In specific embodiments, the emulsifier is present in up to about 22 wt. %.

In specific embodiments, the emulsifier is present in about 3-22 wt. %.

In specific embodiments, the emulsifier is present in at least about 8 wt. %.

In specific embodiments, the emulsifier is present in up to about 18 wt. %.

In specific embodiments, the emulsifier is present in about 8-18 wt. %.

In specific embodiments, the emulsifier is present in about 14 wt. %.

In specific embodiments, the thin film further includes an absorption enhancer.

In specific embodiments, the thin film further includes a flavoring agent.

In specific embodiments, the flavoring agent includes at least one of a liquid flavor extract and a solid flavor extract.

In specific embodiments, the flavoring agent includes a liquid flavor extract.

In specific embodiments, the flavoring agent includes a solid flavor extract.

In specific embodiments, the thin film further includes a sweetener.

In specific embodiments, the sweetener includes at least one of sucralose, acesulfame potassium, ammonium glycyrrhizinate, naringin dihydrochalcone, neohesperidin dihydrochalcone, neotame, erythritol, xylitol, sucrose, sodium saccharine, stevia, alitame, fructose, and aspartame.

In specific embodiments, the sweetener includes at least one of sucralose, acesulfame potassium, and ammonium glycyrrhizinate.

In specific embodiments, the sweetener is present in at least about 1 wt. %.

In specific embodiments, the sweetener is present in up to about 40 wt. %.

In specific embodiments, the sweetener is present in about 1-40 wt. %.

In specific embodiments, the sweetener is present in at least about 6 wt. %.

In specific embodiments, the sweetener is present in up to about 14 wt. %.

In specific embodiments, the sweetener is present in about 6-14 wt. %.

In specific embodiments, the sweetener is present in about 10 wt. %.

In specific embodiments, the thin film further includes a dye or pigment.

In specific embodiments, the dye or pigment includes at least one of FD&C food colorings and vegetable derived food colorings.

In specific embodiments, the dye or pigment includes FD&C food colorings.

In specific embodiments, the dye or pigment includes vegetable derived food colorings.

In specific embodiments, the dye or pigment is present in up to about 0.1 wt. %.

In specific embodiments, the dye or pigment is present in about 0-0.1 wt. %.

In specific embodiments, the dye or pigment is present in at least about 0.01 wt. %.

In specific embodiments, the dye or pigment is present in up to about 0.04 wt. %.

In specific embodiments, the dye or pigment is present in about 0.01-0.04 wt. %.

In specific embodiments, the dye or pigment is present in about 0.2 wt. %.

In specific embodiments, the thin film further includes a preservative.

In specific embodiments, the preservative includes at least one of benzoate salt, sorbate salt, and natamycin.

In specific embodiments, the preservative includes sodium benzoate.

In specific embodiments, the preservative is present in up to about 0.1 wt. %.

In specific embodiments, the preservative is present in about 0-0.1 wt. %.

In specific embodiments, the preservative is present in up to about 0.02 wt. %.

In specific embodiments, the preservative is present in about 0-0.02 wt. %.

In specific embodiments, the thin film further includes a powder coating.

In specific embodiments, the powder coating includes at least one of talc, microcrystalline cellulose, powdered flavoring, and sweetener.

In specific embodiments, the powder coating includes at least one of talc and microcrystalline cellulose.

In specific embodiments, the powder coating includes talc and microcrystalline cellulose.

In specific embodiments, the powder coating is present in up to about 20 wt. %.

In specific embodiments, the powder coating is present in about 0-20 wt. %.

In specific embodiments, the powder coating is present in at least about 5 wt. %.

In specific embodiments, the powder coating is present in up to about 15 wt. %.

In specific embodiments, the powder coating is present in about 5-15 wt. %.

In specific embodiments, the powder coating is present in about 10 wt. %.

In specific embodiments, the active ingredient is present in at least about 20 wt. %.

In specific embodiments, the active ingredient is present in at least about 25 wt. %.

In specific embodiments, the active ingredient is present in at least about 30 wt. %.

In specific embodiments, the active ingredient is present in at least about 35 wt. %.

In specific embodiments, the active ingredient is present in at least about 40 wt. %.

In specific embodiments, the active ingredient is present in up to about 50 wt. %.

In specific embodiments, the active ingredient is present in up to about 45 wt. %.

In specific embodiments, the active ingredient is present in up to about 40 wt. %.

In specific embodiments, the active ingredient is present in up to about 35 wt. %.

In specific embodiments, the active ingredient is present in about 25-45 wt. %.

In specific embodiments, the active ingredient is present in about 25-40 wt. %.

In specific embodiments, the active ingredient is at least partially encapsulated by the lipid.

In specific embodiments, at least about 10 wt. % of the active ingredient is encapsulated by the lipid.

In specific embodiments, at least about 25 wt. % of the active ingredient is encapsulated by the lipid.

In specific embodiments, at least about 35 wt. % of the active ingredient is encapsulated by the lipid.

In specific embodiments, at least about 50 wt. % of the active ingredient is encapsulated by the lipid.

In specific embodiments, at least about 60 wt. % of the active ingredient is encapsulated by the lipid.

In specific embodiments, at least about 75 wt. % of the active ingredient is encapsulated by the lipid.

In specific embodiments, at least about 85 wt. % of the active ingredient is encapsulated by the lipid.

In specific embodiments, at least about 90 wt. % of the active ingredient is encapsulated by the lipid.

In specific embodiments, at least about 95 wt. % of the active ingredient is encapsulated by the lipid.

In specific embodiments, up to about 99 wt. % of the active ingredient is encapsulated by the lipid.

In specific embodiments, up to about 90 wt. % of the active ingredient is encapsulated by the lipid.

In specific embodiments, up to about 75 wt. % of the active ingredient is encapsulated by the lipid.

In specific embodiments, up to about 50 wt. % of the active ingredient is encapsulated by the lipid.

In specific embodiments, the active ingredient is completely encapsulated by the lipid.

In specific embodiments, the lipid encapsulates the active ingredient by the formation of liposomes and/or micelles.

In specific embodiments, the lipid encapsulates the active ingredient by the formation of liposomes.

In specific embodiments, the lipid encapsulates the active ingredient by the formation of unilamellar liposomes.

In specific embodiments, the lipid encapsulates the active ingredient by the formation of multilamellar liposomes.

In specific embodiments, the lipid encapsulates the active ingredient by the formation of micelles.

In specific embodiments, the active ingredient includes one or more antacids.

In specific embodiments, the active ingredient includes one or more H₂-antagonists.

In specific embodiments, the active ingredient includes one or more analgesics.

In specific embodiments, the active ingredient includes calcium carbonate alone or in combination with magnesium hydroxide, and/or aluminum hydroxide.

In specific embodiments, the active ingredient includes one or more antacids in combination with one or more H₂-antagonists.

In specific embodiments, the active ingredient includes one or more opiates or opiate derivatives.

In specific embodiments, the active ingredient includes oxycodone (available as Oxycontin®), ibuprofen, aspirin, acetaminophen, or a combination thereof.

In specific embodiments, the active ingredient includes caffeine.

In specific embodiments, the active ingredient includes one or more anti-diarrheals such as loperamide (available as Imodium®).

In specific embodiments, the active ingredient includes one or more anti-histamines.

In specific embodiments, the active ingredient includes one or more anti-tussives.

In specific embodiments, the active ingredient includes one or more decongestants.

In specific embodiments, the active ingredient includes one or more breath fresheners.

In specific embodiments, the active ingredient includes one or more common active ingredients used alone or in combination for colds, pain, fever, cough, congestion, runny nose and/or allergies, such as acetaminophen, chlorpheniramine maleate, dextromethorphan, pseudoephedrine HCl, and/or diphenhydramine.

In specific embodiments, the active ingredient includes one or more anxiolytics such as alprazolam (available as Xanax®).

In specific embodiments, the active ingredient includes one or more anti-psychotics such as clozopine (available as Clozaril®) and haloperidol (available as Haldol®).

In specific embodiments, the active ingredient includes one or more adrenergic agonists such as clonidine.

In specific embodiments, the active ingredient includes one or more non-steroidal anti-inflammatories (NSAID's) such as dicyclofenac (available as Voltaren®) and etodolac (available as Lodine®).

In specific embodiments, the active ingredient includes one or more anti-histamines such as loratadine (available as Claritin®), astemizole (available as Hismanal®), nabumetone (available as Relafen®), and clemastine (available as Tavist®).

In specific embodiments, the active ingredient includes one or more anti-emetics such as granisetron hydrochloride (available as Kytril®), serotonin 5-HT3 receptor antagonists such as ondansetron (available as Zofran®) and nabilone (available as Cesamet™).

In specific embodiments, the active ingredient includes one or more bronchodilators such as salbutamol (aka albuterol, available as Ventolin®), albuterol sulfate (available as Proventil®).

In specific embodiments, the active ingredient includes one or more anti-depressants such as fluoxetine hydrochloride (available as Prozac®), sertraline hydrochloride (available as Zoloft®), and paroxetine hydrochloride (available as Paxil®).

In specific embodiments, the active ingredient includes one or more anti-migraines such as sumatriptan (available as Imigran®).

In specific embodiments, the active ingredient includes one or more ACE-inhibitors such as enalapril (available as Vasotec®), captopril (available as Capoten®) and lisinopril (available as Prinivil® and Zestril®).

In specific embodiments, the active ingredient includes one or more anti-Alzheimer's agents, such as nicergoline.

In specific embodiments, the active ingredient includes one or more calcium channel blockers (CCBs) such as nifedipine (available as Procardia® and Adalat®), and verapamil hydrochloride (available as Calan®).

In specific embodiments, the active ingredient includes one or more opioid analgesics such as fentanyl (available as Sublimaze®), alfentanil, sufentanil, remifentanil, carfentanil, and lofentanil.

In specific embodiments, the active ingredient includes one or more local anesthetics such as benzocaine (available as Cepacol® and Anbesol®).

In specific embodiments, the active ingredient includes one or more peptide hormones such as insulin.

In specific embodiments, the active ingredient includes one or more oral contraceptives such as estrogen (estradiol) and a progestogen (progestin).

In specific embodiments, the active ingredient includes one or more vaccines such as killed vaccines (e.g., influenza vaccine, cholera vaccine, bubonic plague vaccine, polio vaccine, hepatitis A vaccine, and rabies vaccine); attenuated vaccines (e.g., yellow fever, measles, rubella, and mumps); toxoid vaccines (e.g., tetanus and diphtheria); subunit vaccines (e.g., subunit vaccine against Hepatitis B virus, virus-like particle (VLP) vaccine against human papillomavirus, and the hemagglutinin and neuraminidase subunits of the influenza virus).

In specific embodiments, the active ingredient includes one or more fluoridating agents such as sodium fluoride, sodium monofluorophosphate (MFP), and stannous fluoride.

In specific embodiments, the active ingredient includes one or more stimulants such as caffeine, theobromine, theophylline, yohimbine, and nicotine; energy boosters such as methylxanthines (e.g., caffeine), B vitamins (e.g., Vitamin B12), herbs, guarana, yerba mate, acai, taurine, various forms of ginseng, maltodextrin, inositol, carnitine, creatine, glucuronolactone, ginkgo biloba, bitter orange extract, coenzyme Q10, amino acids (e.g., L-carnitine), bee pollen, royal jelly, green tea extract, spirulina, gotu kola, and glucose.

In specific embodiments, the active ingredient includes one or more sports supplements such as fish oil, dietary protein, creatine, caffeine, glutamine, essential fatty acids (e.g., (alpha-linolenic acid and linoleic acid), prohormones (e.g., chrysin and 4-androstene-3,6,17-trione), and testosterone boosters (e.g., Fenugreek, Eurycoma longifolia, D-Aspartic acid, Boron, L-Carnitine and Tribulus terrestris).

In specific embodiments, the active ingredient includes one or more opioid antidiarrheals such as loperamide (available as Imodium®).

In specific embodiments, the active ingredient includes one or more analgesics such as non-steroidal anti-inflammatory drugs (NSAIDs).

In specific embodiments, the active ingredient includes one or more COX-2 inhibitors such as rofecoxib, celecoxib and etoricoxib.

In specific embodiments, the active ingredient includes one or more opiates such as morphine, diacetylmorphine, codeine, oxycodone, hydrocodone, dihydromorphine, pentazocine, butorphanol, and pethidine.

In specific embodiments, the active ingredient includes at least one of: sildenafil citrate (marketed as Viagra®); tadalafil (marketed as Cialis®); Vitamin D (cholecalciferol); Vitamin C (L-ascorbic acid); testosterone (marketed as Androderm®); fentanyl (marketed as Actiq®); ibuprofen (marketed as Advil®); aspirin (marketed as Bayer®); ranitidine (marketed as Zantac®); loperamide (marketed as Imodium®); acetaminophen (marketed as Tylenol®); ginseng; ondansetron (marketed as Zuplenz®); sucralose; and melatonin (N-acetyl-5-methoxytryptamine).

In specific embodiments, the active ingredient includes sildenafil citrate (marketed as Viagra®).

In specific embodiments, the active ingredient includes tadalafil (marketed as Cialis®).

In specific embodiments, the active ingredient includes Vitamin D (cholecalciferol).

In specific embodiments, the active ingredient includes Vitamin C (L-ascorbic acid).

In specific embodiments, the active ingredient includes testosterone (marketed as Androderm®).

In specific embodiments, the active ingredient includes fentanyl (marketed as Actiq®).

In specific embodiments, the active ingredient includes ibuprofen (marketed as Advil®).

In specific embodiments, the active ingredient includes aspirin (marketed as Bayer®).

In specific embodiments, the active ingredient includes ranitidine (marketed as Zantac®).

In specific embodiments, the active ingredient includes loperamide (marketed as Imodium®).

In specific embodiments, the active ingredient includes acetaminophen (marketed as Tylenol®).

In specific embodiments, the active ingredient includes ginseng.

In specific embodiments, the active ingredient includes ondansetron (marked as Zuplenz®).

In specific embodiments, the active ingredient includes melatonin (N-acetyl-5-methoxytryptamine).

In specific embodiments, the active ingredient includes dietary supplements such as melatonin (N-acetyl-5-methoxytryptamine), vitamins, minerals, fiber, fatty acids, and amino acids.

In specific embodiments, the active ingredient includes one or more electrolytes such as sodium (Na⁺), potassium (K⁺), calcium (Ca²⁺), magnesium (Mg²⁺), chloride (Cl⁻), hydrogen phosphate (HPO₄²⁻), and hydrogen carbonate (HCO³⁻).

In specific embodiments, the active ingredient includes one or more sweeteners (e.g., artificial sweeteners and/or natural sweeteners).

In specific embodiments, the active ingredient includes one or more erectile dysfunction therapies, such as drugs for facilitating blood flow to the penis, and for effecting autonomic nervous activities, such as increasing parasympathetic (cholinergic) and decreasing sympathetic (adrenergic) activities. Useful non-limiting active ingredients include sildenafil, such as Viagra®, tadalafil, such as Cialis®, vardenafil, apomorphines, such as Uprima®, yohimbine hydrochloride such as Aphrodyne®, and alprostadil such as Caverject®.

In specific embodiments, the active ingredient includes one or more H₂-antagonists such as cimetidine, ranitidine hydrochloride, famotidine, nizatidine, ebrotidine, mifentidine, roxatidine, pisatidine and aceroxatidine.

In specific embodiments, the active ingredient includes one or more sweeteners.

In specific embodiments, the active ingredient includes one or more artificial sweeteners such as acesulfame potassium (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides.

In specific embodiments, the active ingredient includes one or more natural sweeteners such as glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

In specific embodiments, the active ingredient includes a low-dose (e.g., 81 mg) of aspirin.

In specific embodiments, the active ingredient includes one or more flavoring agents such as menthol, spearmint, cinnamon, coffee beans, fruit flavor (e.g., cherry flavor, orange flavor, grape flavor, etc.), and coffee flavor.

In specific embodiments, the active ingredient includes one or more amino acids.

In specific embodiments, the active ingredient includes one or more standard amino acids (e.g., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and/or valine).

In specific embodiments, the active ingredient includes one or more non-standard amino acids (e.g., hydroxyproline, hydroxylysine, selenomethionine, selenocysteine, pyrrolysine, lanthionine, 2-aminoisobutyric acid, dehydroalanine, beta alanine (3-aminopropanoic acid), ornithine, citrulline, and/or gamma-aminobutyric acid).

In specific embodiments, the thin film is palatable to a human.

In specific embodiments, the external surfaces of the thin film have a smooth texture.

In specific embodiments, the thin film has a high tensile strength.

In specific embodiments, the thin film is pliable.

In specific embodiments, the thin film is non-sticky to touch.

In specific embodiments, the thin film does not readily stick to another thin film.

In specific embodiments, the thin film is relatively soft to touch.

In specific embodiments, the thin film has a chewable configuration.

In specific embodiments, the thin film has a resilient configuration.

In specific embodiments, the thin film has an elastic or malleable configuration.

In specific embodiments, the thin film has a ductile property.

In specific embodiments, the thin film further includes a bitter blocker.

In specific embodiments, the thin film further includes a powder coating present on at least one external surface of the thin film.

In specific embodiments, the thin film further includes a powder coating present on two opposing external surfaces of the thin film.

In specific embodiments, the thin film is white.

In specific embodiments, the thin film is black.

In specific embodiments, the thin film is yellow.

In specific embodiments, the thin film is blue.

In specific embodiments, the thin film is green.

In specific embodiments, the thin film is pink.

In specific embodiments, the thin film is red.

In specific embodiments, the thin film is orange.

In specific embodiments, the thin film is violet.

In specific embodiments, the thin film is indigo.

In specific embodiments, the thin film is brown.

In specific embodiments, the thin film has at least one of printed indicia, dosage, instructions, ingredients, a logo, and a trademark located on a surface therein.

In specific embodiments, the method of preparing a thin film is carried out in the order indicated.

In specific embodiments, each of the steps of the method of preparing a thin film is carried out in the order indicated.

In specific embodiments, the mixing includes blending.

In specific embodiments, the mixing is carried out at a temperature of about 130-140° F. (54.4-60° C.).

In specific embodiments, the contacting of the active ingredient with the uniform first mixture further includes contacting at least one of a sweetener, flavoring agent, and dye or pigment with the uniform first mixture.

In specific embodiments, the contacting of the binder with the thickened second mixture includes initially contacting a non-sticky binder with the thickened second mixture, and subsequently contacting a sticky binder with the thickened second mixture.

In specific embodiments, the contacting of the binder with the thickened second mixture includes initially contacting microcrystalline cellulose with the thickened second mixture, and subsequently contacting pectin with the thickened second mixture.

In specific embodiments, the condensing includes heating at a temperature of at least about 75° F. (23.9° C.).

In specific embodiments, the condensing includes heating at a temperature of at least about 100° F. (37.8° C.).

In specific embodiments, the condensing includes heating at a temperature of at least about 125° F. (51.7° C.).

In specific embodiments, the condensing includes heating at a temperature of at least about 150° F. (65.6° C.).

In specific embodiments, the condensing includes heating at a temperature of up to about 200° F. (93.4° C.).

In specific embodiments, the condensing includes heating at a temperature of up to about 150° F. (65.6° C.).

In specific embodiments, the condensing includes heating at a temperature of about 75° F. (23.9° C.) to about 200° F. (93.4° C.).

In specific embodiments, the condensing includes heating at a temperature of about 75° F. (23.9° C.) to about 175° F. (79.4° C.).

In specific embodiments, the condensing includes heating at a temperature of about 75° F. (23.9° C.) to about 150° F. (65.6° C.).

In specific embodiments, the condensing is carried out for at least about 10 minutes.

In specific embodiments, the condensing is carried out for at least about 30 minutes.

In specific embodiments, the condensing is carried out for at least about 1 hour.

In specific embodiments, the condensing is carried out for at least about 2 hours.

In specific embodiments, the condensing is carried out for at least about 6 hours.

In specific embodiments, the condensing is carried out for at least about 8 hours.

In specific embodiments, the condensing is carried out for at least about 12 hours.

In specific embodiments, the condensing is carried out for at least about 18 hours.

In specific embodiments, the condensing is carried out for at least about 24 hours.

In specific embodiments, the condensing is carried out for at least about 48 hours.

In specific embodiments, the condensing includes heating with passive convection.

In specific embodiments, the condensing includes heating with active convection.

In specific embodiments, the condensing is carried out in vacuo.

In specific embodiments, the method of preparing a thin film further includes contacting a powder coating with at least one external surface of the thin film.

In specific embodiments, the method of preparing a thin film further includes contacting a powder coating with two opposing external sides of the thin film.

In specific embodiments, the system includes at least about 2 thin films.

In specific embodiments, the system includes at least about 5 thin films.

In specific embodiments, the system includes at least about 10 thin films.

In specific embodiments, the system includes at least about 25 thin films.

In specific embodiments, each of the multiple thin films of the system independently has a dimension of at least 15 mm×25 mm.

In specific embodiments, each of the multiple thin films of the system independently has a dimension of up to 35 mm×50 mm.

In specific embodiments, each of the multiple thin films of the system independently has a dimension of 23 mm×38 mm, ±5 mm.

In specific embodiments, each of the multiple thin films of the system independently has a dimension of 23 mm×38 mm, ±3 mm.

In specific embodiments, each of the multiple thin films of the system independently has a thickness of about 0.01 mm to about 20 mm.

In specific embodiments, each of the multiple thin films of the system independently has a thickness of at least about 0.01 mm.

In specific embodiments, each of the multiple thin films of the system independently has a thickness of up to about 20 mm.

In specific embodiments, each of the multiple thin films of the system independently has a thickness of about 0.03 mm to about 1 mm.

In specific embodiments, each of the multiple thin films of the system independently weighs about 10 mg to about 80 mg.

In specific embodiments, each of the multiple thin films of the system independently weighs at least about 10 mg.

In specific embodiments, each of the multiple thin films of the system independently weighs about up to about 80 mg.

In specific embodiments, each of the multiple thin films of the system independently weighs about 20 mg to about 70 mg.

In specific embodiments, each of the multiple thin films of the system independently weighs at least about 30 mg.

In specific embodiments, each of the multiple thin films of the system independently weighs up to at least about 400 mg.

In specific embodiments, each of the multiple thin films of the system independently weighs about 30 mg to about 400 mg.

In specific embodiments, each of the multiple thin films of the system independently weighs about 60 mg to about 300 mg.

### Enumerated Embodiments

Specific enumerated embodiments [1] to [95] provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

[1.] A thin film that includes:

(a) solvent,

(b) binder,

(c) lipid,

(d) emulsifier, and

(e) active ingredient.

[2.] The thin film of the above embodiment, wherein the solvent includes at least one of water and ethanol.
[3.] The thin film of any one of the above embodiments, wherein the solvent is present in about 2-24 wt. %.
[4.] The thin film of any one of the above embodiments, wherein the solvent is present in about 4-12 wt. %.
[5.] The thin film of any one of the above embodiments, wherein the solvent is present in about 8 wt. %.
[6.] The thin film of any one of the above embodiments, wherein the binder includes at least one of pectin, microcrystalline cellulose, xanthan gum, locust bean gum, guar gum, gum arabic, gum tragacanth, gum karaya, beta glucan, glucomannan, tapioca starch, carrageenan, xanthan gum, gellan gum, alginic acid or sodium alginate, konjac gum, tara gum, chitosan, agar, maltodextrin, polyvinyl alcohol, pullulan, polycarbophil, povidone, hydroxypropyl methylcellulose, hydroxypropyl cellulose, hydroxypropyl starch, polyacrylic acid, and polyethylene glycol.
[7.] The thin film of any one of the above embodiments, wherein the binder is acidic.
[8.] The thin film of any one of the above embodiments, wherein the binder is present in about 4-50 wt. %.
[9.] The thin film of any one of the above embodiments, wherein the binder is present in about 10-36 wt. %.
[10.] The thin film of any one of the above embodiments, wherein the binder is present in about 25 wt. %.
[11.] The thin film of any one of the above embodiments, wherein the lipid includes at least one of almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, Shea butter, soybean oil, sunflower oil, walnut oil, and watermelon seed oil.
[12.] The thin film of any one of the above embodiments, wherein the lipid is present in about 4-22 wt. %.
[13.] The thin film of any one of the above embodiments, wherein the lipid is present in about 10-18 wt. %.
[14.] The thin film of any one of the above embodiments, wherein the lipid is present in about 14.8 wt. %.
[15.] The thin film of any one of the above embodiments, wherein the emulsifier includes at least one of glycerin, propylene glycol, and polyethylene glycol.
[16.] The thin film of any one of the above embodiments, wherein the emulsifier is present in about 3-22 wt. %.
[17.] The thin film of any one of the above embodiments, wherein the emulsifier is present in about 8-18 wt. %.
[18.] The thin film of any one of the above embodiments, wherein the emulsifier is present in about 14 wt. %.
[19.] The thin film of any one of the above embodiments, further including a flavoring agent.
[20.] The thin film of any one of the above embodiments, further including a flavoring agent, including at least one of a liquid flavor extract and a solid flavor extract.
[21.] The thin film of any one of the above embodiments, further including a sweetener.
[22.] The thin film of any one of the above embodiments, further including a sweetener selected from at least one of sucralose, acesulfame potassium, ammonium glycyrrhizinate, naringin dihydrochalcone, neohesperidin dihydrochalcone, neotame, erythritol, xylitol, sucrose, sodium saccharine, stevia, alitame, fructose, and aspartame.
[23.] The thin film of any one of the above embodiments, further including a sweetener, present in about 1-40 wt. %.
[24.] The thin film of any one of the above embodiments, further including a sweetener, present in about 6-14 wt. %.
[25.] The thin film of any one of the above embodiments, further including a sweetener, present in about 10 wt. %.
[26.] The thin film of any one of the above embodiments, further including a dye or pigment.
[27.] The thin film of any one of the above embodiments, further including a dye or pigment selected from at least one of FD&C food colorings and vegetable derived food colorings.
[28.] The thin film of any one of the above embodiments, further including a dye or pigment, present in about 0-0.1 wt. %.
[29.] The thin film of any one of the above embodiments, further including a dye or pigment, present in about 0.01-0.04 wt. %.
[30.] The thin film of any one of the above embodiments, further including a dye or pigment, present in about 0.2 wt. %.
[31.] The thin film of any one of the above embodiments, further including a preservative.
[32.] The thin film of any one of the above embodiments, further including a preservative selected from at least one of benzoate salt, sorbate salt, and natamycin.
[33.] The thin film of any one of the above embodiments, further including a preservative, present in about 0-0.1 wt. %.
[34.] The thin film of any one of the above embodiments, further including a preservative, present in about 0-0.02 wt. %.
[35.] The thin film of any one of the above embodiments, further including a powder coating.
[36.] The thin film of any one of the above embodiments, further including a powder coating selected from at least one of talc and microcrystalline cellulose.
[37.] The thin film of any one of the above embodiments, further including a powder coating, present in about 0-20 wt. %.
[38.] The thin film of any one of the above embodiments, further including a powder coating, present in about 5-15 wt. %.
[39.] The thin film of any one of the above embodiments, further including a powder coating, present in about 10 wt. %.
[40.] The thin film of any one of the above embodiments, wherein the active ingredient is present in at least about 25 wt. %.
[41.] The thin film of any one of the above embodiments, wherein the active ingredient is present in at least about 30 wt. %.
[42.] The thin film of any one of the above embodiments, wherein the active ingredient is present in at least about 35 wt. %.
[43.] The thin film of any one of the above embodiments, wherein the active ingredient is present in at least about 40 wt. %.
[44.] The thin film of any one of the above embodiments, wherein the active ingredient is present in about 25-40 wt. %.
[45.] The thin film of any one of the above embodiments, wherein the active ingredient is at least partially encapsulated by the lipid.
[46.] The thin film of any one of the above embodiments, wherein at least about 25 wt. % of the active ingredient is encapsulated by the lipid.
[47.] The thin film of any one of the above embodiments, wherein at least about 50 wt. % of the active ingredient is encapsulated by the lipid.
[48.] The thin film of any one of the above embodiments, wherein at least about 75 wt. % of the active ingredient is encapsulated by the lipid.
[49.] The thin film of any one of the above embodiments, wherein the active ingredient is completely encapsulated by the lipid.
[50.] The thin film of any one of the above embodiments, wherein the active ingredient includes at least one of: sildenafil citrate (marketed as Viagra®); tadalafil (marketed as Cialis®); Vitamin D (cholecalciferol); Vitamin C (L-ascorbic acid); testosterone (marketed as Androderm®); fentanyl (marketed as Actiq®); ibuprofen (marketed as Advil®); aspirin (marketed as Bayer®); ranitidine (marketed as Zantac®); loperamide (marketed as Imodium®); acetaminophen (marketed as Tylenol®); aripiprazole (marketed as Abilify®); famotidine (marketed as Pepcid®); morphine (marketed as Oramorph®); ginseng; ondansetron (marketed as Zuplenz®); sucralose; caffeine, amino acid, and melatonin (N-acetyl-5-methoxytryptamine).
[51.] The thin film of any one of the above embodiments, which is palatable to a human.
[52.] The thin film of any one of the above embodiments, wherein the external surfaces have a smooth texture.
[53.] The thin film of any one of the above embodiments, which has a high tensile strength.
[54.] The thin film of any one of the above embodiments, which is pliable.
[55.] The thin film of any one of the above embodiments, which is non-sticky to touch.
[56.] The thin film of any one of the above embodiments, which does not readily stick to another thin film.
[57.] The thin film of any one of the above embodiments, which is relatively soft to touch.
[58.] The thin film of any one of the above embodiments, having a chewable configuration.
[59.] The thin film of any one of the above embodiments, having a resilient configuration.
[60.] The thin film of any one of the above embodiments, having an elastic or malleable configuration.
[61.] The thin film of any one of the above embodiments, including a ductile property.
[62.] The thin film of any one of the above embodiments, further including a bitter blocker.
[63.] The thin film of any one of the above embodiments, further including a powder coating present on at least one external surface of the thin film.
[64.] The thin film of any one of the above embodiments, further including a powder coating present on two opposing external surfaces of the thin film.
[65.] The thin film of any one of embodiments [63]-[64], wherein the powder coating includes at least one of talc and microcrystalline cellulose.
[66.] The thin film of any one of the above embodiments, wherein the thin film includes:

(a) about 2-24 wt. % solvent,

(b) about 4-50 wt. % binder,

(c) about 0-10 wt. % flavoring agent,

(d) about 1-40 wt. % sweetener,

(e) about 4-22 wt. % lipid,

(f) about 3-22 wt. % emulsifier,

(g) about 0-1.0 wt. % dye or pigment,

(h) about 0-0.1 wt. % preservative,

(i) up to about 65 wt. % active ingredient, and

(j) about 0-20 wt. % powder coating.

[67.] The thin film of any one of the above embodiments, wherein the thin film includes:

(a) about 4-12 wt. % solvent,

(b) about 10-36 wt. % binder,

(c) about 2-4 wt. % flavoring agent,

(d) about 6-14 wt. % sweetener,

(e) about 10-18 wt. % lipid,

(f) about 8-18 wt. % emulsifier,

(g) about 0.01-0.04 wt. % dye or pigment,

(h) about 0-0.02 wt. % preservative,

(i) up to about 40 wt. % active ingredient, and

(j) about 5-15 wt. % powder coating.

[68.] The thin film of any one of the above embodiments, wherein the thin film includes:

(a) about 8 wt. % solvent,

(b) about 25 wt. % binder,

(c) about 4 wt. % flavoring agent,

(d) about 9.5 wt. % sweetener,

(e) about 14.8 wt. % lipid,

(f) about 14 wt. % emulsifier,

(g) about 0.02 wt. % dye or pigment,

(h) about 33 wt. % active ingredient, and

(i) about 10 wt. % powder coating.

[69.] A method of preparing a thin film, the method includes:

(a) mixing a lipid, emulsifier, and solvent to provide a uniform first mixture;

(b) contacting an active ingredient with the uniform first mixture to provide a thickened second mixture;

(c) contacting a binder with the thickened second mixture to provide a slurry; and

(d) hot extruding, casting, and condensing the slurry to provide the thin film; or cooling, shearing, mixing, casting, and condensing the slurry to provide the thin film.

[70.] The method of the above embodiment, wherein the thin film obtained is a thin film as described in any one of the above embodiments (e.g., any one of embodiments [1]-[69]).
[71.] The method of any one of the above embodiments, wherein the mixing includes blending.
[72.] The method of any one of the above embodiments, wherein the mixing is carried out at a temperature of about 130-140° F. (54.4-60° C.).
[73.] The method of any one of the above embodiments, wherein the contacting of the active ingredient with the uniform first mixture further includes contacting at least one of a sweetener, flavoring agent, and dye or pigment with the uniform first mixture.
[74.] The method of any one of the above embodiments, wherein the contacting of the binder with the thickened second mixture includes initially contacting a non-sticky binder with the thickened second mixture, and subsequently contacting a sticky binder with the thickened second mixture.
[75.] The method of any one of the above embodiments, wherein the contacting of the binder with the thickened second mixture includes initially contacting microcrystalline cellulose with the thickened second mixture, and subsequently contacting pectin with the thickened second mixture.
[76.] The method of any one of the above embodiments, wherein the condensing includes heating at a temperature of at least about 50° F. (10° C.).
[77.] The method of any one of the above embodiments, wherein the condensing is carried out for at least about 10 minutes.
[78.] The method of any one of the above embodiments, wherein the condensing includes heating with passive convection.
[79.] The method of any one of the above embodiments, wherein the condensing includes heating with active convection.
[80.] The method of any one of the above embodiments, wherein the condensing is carried out in vacuo.
[81.] The method of any one of the above embodiments, further including contacting a powder coating with at least one external surface of the thin film.
[82.] The method of any one of the above embodiments, further including contacting a powder coating with two opposing external sides of the thin film.
[83.] The method of any one of embodiments [81]-[82], wherein the powder coating includes at least one of talc and microcrystalline cellulose.
[84.] A method of administering a thin film to a mucosal surface or wound of an animal (e.g., human), the method includes contacting the mucosal surface or wound with the thin film described herein.
[85.] A method of administering an active ingredient to a mucosal surface or wound of an animal (e.g., human), the method includes contacting the mucosal surface or wound with a thin film described herein.
[86.] A method of treating a disease or disorder in an animal (e.g., human), the method includes contacting the mucosal surface or wound of a patient in need of such treatment with a thin film described herein.
[87.] A method of introducing to a liquid (or liquid containing substance) a thin film, the method includes contacting the liquid (or liquid containing substance) with a thin film described herein, and allowing it to dissolve.
[88.] A method of preparing a liquid dosage form of an active, the method includes contacting a liquid (or liquid containing substance) with a thin film described herein, and allowing it to dissolve.
[89.] A method of flavoring a beverage or food product, the method includes contacting the beverage or food product with a thin film described herein, and allowing it to dissolve.
[90.] A method of adding at least one of a sweetener, electrolytes, nutrients, nutraceuticals, active ingredient, vitamins, and protein to a beverage or food product, the method includes contacting the beverage or food product with a thin film described herein, and allowing it to dissolve.
[91.] A system that includes:

(a) multiple thin films, each in direct contact with at least one other thin film, and each independently described in any one of the above embodiments (e.g., any one of embodiments [1]-[68]);

(b) packaging material enclosing the multiple thin films; and

(c) printed indicia located on the packaging material; wherein the multiple thin films do not readily stick to another.

[92.] The system of the above embodiment, including at least about 5 thin films.
[93.] The system of any one of the above embodiments, including up to about 25 thin films.
[94.] The system of any one of the above embodiments, wherein each of the multiple thin films independently has a dimension of 23 mm×38 mm, ±3 mm.
[95.] The system of any one of the above embodiments, wherein each of the multiple thin films is independently prepared as described in any one of the above embodiments (e.g., any one of embodiments [69]-[90]).

The invention will now be described by the following non-limiting examples.

### EXAMPLES

### Example 1

### Preparation of Thin Film with an Encapsulated Active Ingredient

Liposomes can be prepared by using a phospholipid like hydroxylated lecithin, an oil such as deodorized cocoa butter, water, and glycerin. These substances are mixed (warm), so the cocoa butter is melted, using a high shear blender or a hand-held mixer. To this is added the active ingredient(s), whether they are water-soluble or fat-soluble. A high shear blender can be employed. The active ingredient(s) can be incorporated into the layers of the forming liposomes. After the formation of the active impregnated liposomes, the following substances can be added: flavorings, sweeteners, colors, non-sticky binders (e.g., microcrystalline cellulose), and finally a sticky binder (e.g., pectin). This slurry is now a colloid consisting of liposomes dispersed in an aqueous hydrogel.

The slurries are typically 60-80% water. They can be spread onto glass in the laboratory or onto PET (polyethylene terephthalate) film in the factory and cooked at 60-105° C. both in laboratory and factory settings. The slurries can be hardened into flexible films that are 4-22% water when cooking is complete. Further cooking leads to brittle and eventually charred products. Laboratory films are typically cut and packaged as soon as they are out of the oven. The hardened films from the factory are cured for 1-4 days then slit, cut, and packaged. The films cannot be over-cured, so sometimes longer cure times happen due to time and availability of working staff Driving off of water from a warm and pliable slurry is believed to yield metamorphosed liposomal structures now suspended in close proximity in a solid wafer film. Curing completes the metamorphosis. Without being bound to any particular theory, there is believed to be a plurality of liposomal structures, and even multilayer lipid bilayer sheets in the final products.

### Example 2

### Preparation of Caffeine-Containing Oral Thin Film

Calculations were done such that the active ingredient was approximately 33% of the final oral thin film. The final weight was determined to be approximately 110 mg for a 23×38 mm oral thin film.

14.0 g glycerin was placed into a 50 mL glass beaker. To this was added 7.0 g hydroxylated lecithin and 7.8 g deodorized cocoa butter. 20 mL water was added and heated in microwave oven for approximately 35 seconds so that the water was warm enough to melt the cocoa butter (approximately 49° C.). The mixture was stirred manually to insure complete melting of the cocoa butter then poured into a blender. Additional 245 mL warm water (approximately 46° C.) was added to the blender and the mixture was blended for approximately 20 seconds. With blending, the following ingredients were added: 33.0 g caffeine, 6.0 g sucralose, 3.0 g acesulfame potassium, 0.5 g ammonium glycyrrhizinate, 2.0 g Crystal White® flavoring, 0.5 g sodium chloride, 4.0 g microcrystalline cellulose, 12 mg FD&C yellow #5, 6.o mg FD&C blue #1. 26.0 g pectin was added slowly and stirred for about 3 minutes until the mixture goes from a granular look to a smooth glossy look. This material was poured onto a glass plate and a steel rod with 20 gauge wire depth gauges was dragged across the plate creating a uniform film on the plate. Excess slurry was scraped back into the slurry batch. The coated glass plate was placed in an oven at 75° C. with passive convection for 20-30 minutes. Once visibly dry, the film was cut using 23×38 mm templates. Flavored powder coating consisting of 45% talc, 50% microcrystalline cellulose, 1% micronized sucralose, and 4% flavoring powder was sprinkled on the film and the individual films were removed from the glass plate with a razor blade.

### Example 3

### Exemplary Formulations, Oral Thin Film

| | Amount | | Amount | | Amount | |
|---|---|---|---|---|---|---|
| Ingredient | present | | present | | present | |
| solvent | 2-24 | wt. % | 4-12 | wt. % | 8 | wt. % |
| binder | 4-50 | wt. % | 10-36 | wt. % | 25 | wt. % |
| flavoring agent | 0-10 | wt. % | 2-4 | wt. % | 4 | wt. % |
| sweetener | 1-40 | wt. % | 6-14 | wt. % | 9.5 | wt. % |
| lipid | 4-22 | wt. % | 10-18 | wt. % | 14.8 | wt. % |
| emulsifier | 3-22 | wt. % | 8-18 | wt. % | 14 | wt. % |
| dye or pigment | 0-1.0 | wt. % | 0.01-0.04 | wt. % | 0.02 | wt. % |
| preservative | 0-0.1 | wt. % | 0-0.02 | wt. % | 0 | wt. % |
| active ingredient | up to about | | up to about | | about | |
| | 65 | wt. % | 40 | wt. % | 33 | wt. % |
| powder coating | 0-20 | wt. % | 5-15 | wt. % | 10 | wt. % |

Note:
In the above formulations, the finished product may include about 8-10 wt. % moisture.

### Example 4

### Exemplary Formulations, Oral Thin Film

| | Amount | | Amount | | Amount | |
|---|---|---|---|---|---|---|
| Ingredient | present | | present | | present | |
| water | 2-24 | wt. % | 4-12 | wt. % | 8 | wt. % |
| pectin and | 4-50 | wt. % | 10-36 | wt. % | 25 | wt. % |
| microcrystalline cellulose | | | | | | |
| liquid and/or solid flavor | 0-10 | wt. % | 2-4 | wt. % | 4 | wt. % |
| extracts | | | | | | |
| sucralose, acesulfame | 1-40 | wt. % | 6-14 | wt. % | 9.5 | wt. % |
| potassium, and/or | | | | | | |
| ammonium | | | | | | |
| glycyrrhizinate | | | | | | |
| hydroxylated lecithin | 4-22 | wt. % | 10-18 | wt. % | 14.8 | wt. % |
| and/or deodorized cocoa | | | | | | |
| butter | | | | | | |
| glycerin | 3-22 | wt. % | 8-18 | wt. % | 14 | wt. % |
| FD&C food colorings | 0-1.0 | wt. % | 0.01-0.04 | wt. % | 0.02 | wt. % |
| sodium benzoate | 0-0.1 | wt. % | 0-0.02 | wt. % | 0 | wt. % |
| active ingredient | up to about | | up to about | | about | |
| | 65 | wt. % | 40 | wt. % | 33 | wt. % |
| talc, microcrystalline | 0-20 | wt. % | 5-15 | wt. % | 10 | wt. % |

cellulose, powdered
flavoring, and/or
sweetener

Note:
In the above formulations, the finished product may include about 8-10 wt. % moisture.

All publications, patents, and published patent applications disclosed herein are incorporated herein by reference in their entirety. While in the foregoing specification this invention (as defined by the issued claims) invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A thin film comprising:
(a) about 2-24 wt. % solvent,
(b) about 4-50 wt. % binder,
(c) about 0-10 wt. % flavoring agent,
(d) about 1-40 wt. % sweetener,
(e) about 4-22 wt. % lipid,
(f) about 3-22 wt. % emulsifier,
(g) about 0-1.0 wt. % dye or pigment,
(h) about 0-0.1 wt. % preservative,
(i) active ingredient present in at least about 25 wt. % and up to about 65 wt. %, and
(j) about 0-20 wt. % powder coating.

2. The thin film of claim 1, wherein the solvent is present in about 4-12 wt. %.

3. The thin film of claim 1, wherein the binder comprises at least one of pectin, microcrystalline cellulose, xanthan gum, locust bean gum, guar gum, gum arabic, gum tragacanth, gum karaya, beta glucan, glucomannan, tapioca starch, carrageenan, xanthan gum, gellan gum, alginic acid or sodium alginate, konjac gum, tara gum, chitosan, agar, maltodextrin, polyvinyl alcohol, pullulan, polycarbophil, povidone, hydroxypropyl methylcellulose, hydroxypropyl cellulose, hydroxypropyl starch, polyacrylic acid, and polyethylene glycol.

4. The thin film of claim 1, wherein the binder is present in about 10-36 wt. %.

5. The thin film of claim 1, wherein the lipid comprises at least one of almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, marula oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, Shea butter, soybean oil, sunflower oil, walnut oil, and watermelon seed oil.

6. The thin film of claim 1, wherein the lipid is present in about 10-18 wt. %.

7. The thin film of claim 1, wherein the emulsifier comprises at least one of glycerin, propylene glycol, and polyethylene glycol.

8. The thin film of claim 1, wherein the emulsifier is present in about 8-18 wt. %.

9. The thin film of claim 1, wherein the sweetener comprises at least one of sucralose, acesulfame potassium, ammonium glycyrrhizinate, naringin dihydrochalcone, neohesperidin dihydrochalcone, neotame, erythritol, xylitol, sucrose, sodium saccharine, stevia, alitame, fructose, and aspartame.

10. The thin film of claim 1, wherein the sweetener is present in about 6-14 wt. %.

11. The thin film of claim 1, wherein the dye or pigment is present in about 0.01-0.04 wt. %.

12. The thin film of claim 1, wherein the preservative is selected from at least one of benzoate salt, sorbate salt, and natamycin.

13. The thin film of claim 1, wherein the powder coating is selected from at least one of talc and microcrystalline cellulose.

14. The thin film of claim 1, wherein the powder coating is present in about 5-15 wt. %.

15. The thin film of claim 1, wherein the active ingredient is present in about 25-40 wt. %.

16. The thin film of claim 1, wherein the active ingredient is at least partially encapsulated by the lipid.

17. The thin film of claim 1, wherein at least about 75 wt. % of the active ingredient is encapsulated by the lipid.

18. The thin film of claim 1, wherein the active ingredient comprises at least one of sildenafil citrate, tadalafil, vitamin D (cholecalciferol), vitamin C (L-ascorbic acid), testosterone, fentanyl, ibuprofen, aspirin, ranitidine, loperamide, acetaminophen, aripiprazole, famotidine, morphine, ginseng, ondansetron, sucralose, caffeine, amino acid, and melatonin (N-acetyl-5-methoxytryptamine).

19. The thin film of claim 1, further comprising a powder coating present on at least one external surface of the thin film.

20. The thin film of claim 1, further comprising a powder coating present on two opposing external surfaces of the thin film.

21. The thin film of claim 1, further comprising a powder coating present on two opposing external surfaces of the thin film, wherein the powder coating comprises at least one of talc and microcrystalline cellulose.

22. The thin film of claim 1, wherein the active ingredient is present in about 25-45 wt. %.

23. The thin film of claim 1, wherein the active ingredient is present in at least about 30 wt. %.

24. The thin film of claim 1, wherein the active ingredient is present in at least about 35 wt. %.

25. The thin film of claim 1, wherein the active ingredient is present in at least about 40 wt. %.
